(19) United States
(12) Patent Application Publication
(10) Pub. No.: US 2025/0282707 A1
(43) Pub. Date: Sep. 11, 2025

(54) NOVEL KETONE ESTER COMPOUNDS

(71) Applicant: Buck Institute for Research on Aging, Novato, CA (US)

(72) Inventors: Brianna J. Stubbs, Novato, CA (US); John C. Newman, Novato, CA (US); Eric Verdin, Novato, CA (US)

(21) Appl. No.: 18/861,309

(22) PCT Filed: Apr. 26, 2023
(86) PCT No.: PCT/US2023/020036

§ 371 Date: Oct. 29, 2024

Related U.S. Application Data
(60) Provisional application No. 63/336,945, filed on Apr. 29, 2022.

(51) Int. Cl. C07C 69/675; C07C 229/24

(52) U.S. Cl. CPC C07C 69/675; C07C 229/24

(57) ABSTRACT

Ester compounds are provided herein that when administered to a mammal (e.g., to a human) are hydrolyzed to produce 1) ketone bodies or immediate precursors to ketone bodies; 2) lactate or immediate precursors to lactate; and/or 3) propionyl 1-carnitine. These three classes of metabolites are known to positively influence function of the heart and the compounds are believed to be useful, inter alia, in the treatment and/or prophylaxis of congestive heart failure.

### CROSS-REFERENCE TO RELATED APPLICATIONS

This application claims benefit of and priority to U.S. Ser. No. 63/336,945, filed on Apr. 29, 2022, which is incorporated herein by reference in its entirety for all purposes.

### INTRODUCTION

Ketogenic diets and ketone bodies are of interest for the treatment of a variety of human disorders including epilepsy, dementia and diseases of aging. Ketone bodies are small compounds created from fat that serve as a substitute for sugar when the body's energy stores are depleted, such as when fasting or during strenuous exercise. Ketogenic diets stimulate the production of ketone bodies by containing very little sugar or other carbohydrates. The primary ketone bodies in humans are acetoacetate (AcAc) and β-hydroxybutyrate (BHB), in particular, the R-enantiomer of BHB. Ketogenic diets are used clinically as a therapy for epilepsy, but they are often difficult to adhere to for long periods of time. The extremely high fat content (and low carbohydrate content) can make foods of a ketogenic diet unpalatable, and sometimes cause gastrointestinal problems, kidney stones, high cholesterol, and other side effects.

Betahydroxybutyrate (BHB) is a metabolic intermediate that is a currency for generating cellular energy, but also has several signaling functions separate from energy production. Either or both of the energy and signaling functions may be important for BHB's effects on human disease. During times of scarce glucose, for example during fasting or strenuous exercise, BHB is the currency by which energy stored in adipose tissue is turned into fuel that can be used by cells throughout the body to sustain their functions. Fat mobilized from adipose tissue is transported to the liver and converted into BHB. BHB circulates in the blood to all tissue. After being absorbed into a cell, BHB is broken down in the mitochondria to generate acetyl-CoA that is further metabolized into ATP. This is the canonical “energy currency” function of BHB.

In addition, BHB is believed to have several signaling functions. Most of these are independent of its function as an energy currency, in that they are actions of the BHB molecule itself and are not generally secondary effects of its metabolism into acetyl-CoA and ATP. Signaling functions may include, but are not limited to: 1) inhibition of class I and IIa histone deacetylases, with resulting changes in histone modifications and gene expression, as well as changes in acetylation state and activity of non-histone proteins; 2) metabolism into acetyl-CoA results in increased cellular production of acetyl-coA to serve as substrate for acetyltransferase enzymes, resulting in similar changes in histone and non-histone protein acetylation as deacetylase inhibition; 3) covalent attachment to histones and possibly other proteins in the form of lysine-β-hydroxybutyrylation, which may have similar effects as lysine-acetylation; 4) binding and activation of the hydroxycarboxylic acid receptor 2 (HCAR2) receptor with resultant alterations in adipose tissue metabolism; 5) binding and inhibition of free fatty acid receptor 3 (FFAR3) receptor with resultant changes in sympathetic nervous system activation and whole-body metabolic rate; and 6) inhibition of the NOD-like receptor 3 (NLRP3) inflammasome.

### SUMMARY

Various embodiments provided herein may include, but need not be limited to, one or more of the following:

Embodiment 1: A compound according to Formula I or Formula II:

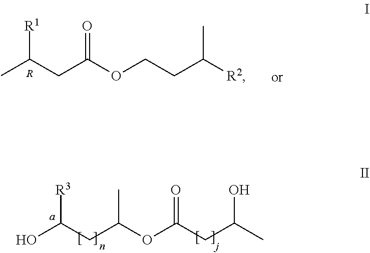

wherein: R¹ is OH or C(4-14) saturated fatty acid; R² is OH or C(4-14) saturated fatty acid; at least one of R¹ or R² is said saturated fatty acid; R³ is H, CH₃, or O; n is 0 or 1; j is 0 or 1; and when R³ is O, a is a double bond.

Embodiment 2: The compound of embodiment 1, wherein said compound comprises a compound according to Formula I:

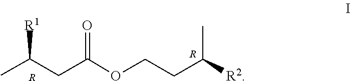

Embodiment 3: The compound according to any one of embodiments 1-2, wherein R¹ is OH.

Embodiment 4: The compound of embodiment 3, wherein R² is a C(4-10) fatty acid.

Embodiment 5: The compound of embodiment 4, wherein R² is a C(4-6) fatty acid.

Embodiment 6: The compound of embodiment 3, wherein R² is a C6 fatty acid.

Embodiment 7: The compound of embodiment 3, wherein R² is a C8 fatty acid.

Embodiment 8: The compound of embodiment 3, wherein R² is a C10 fatty acid.

Embodiment 9: The compound of embodiment 3, wherein R² is a C12 fatty acid.

Embodiment 10: The compound of embodiment 5, wherein said compound is a compound according to the formula:

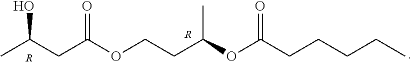

Embodiment 11: The compound according to any one of embodiments 1-2, wherein R² is OH.

Embodiment 12: The compound of embodiment 11, wherein R¹ is a C(4-10) fatty acid.

Embodiment 13: The compound of embodiment 12, wherein R¹ is a C(4-6) fatty acid.

Embodiment 14: The compound of embodiment 12, wherein R¹ is a C6 fatty acid.

Embodiment 15: The compound of embodiment 12, wherein R¹ is a C8 fatty acid.

Embodiment 16: The compound of embodiment 12, wherein R¹ is a C10 fatty acid.

Embodiment 17: The compound of embodiment 12, wherein R¹ is a C12 fatty acid.

Embodiment 18: The compound of embodiment 13, wherein said compound is a compound according to the formula:

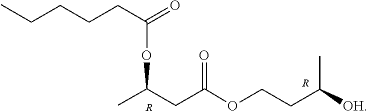

Embodiment 19: The compound according to any one of embodiments 1-2, wherein: R¹ is a C(4-14) saturated fatty acid; and R² is a C(4-14) saturated fatty acid.

Embodiment 20: The compound of embodiment 19, wherein R¹ is a C4 saturated fatty acid.

Embodiment 21: The compound of embodiment 19, wherein R¹ is a C6 saturated fatty acid.

Embodiment 22: The compound of embodiment 19, wherein R¹ is a C8 saturated fatty acid.

Embodiment 23: The compound of embodiment 19, wherein R¹ is a C10 saturated fatty acid.

Embodiment 24: The compound of embodiment 19, wherein R¹ is a C12 saturated fatty acid.

Embodiment 25: The compound according to any one of embodiments 19-24, wherein R² is a C4 fatty acid.

Embodiment 26: The compound according to any one of embodiments 19-24, wherein R² is a C6 fatty acid.

Embodiment 27: The compound according to any one of embodiments 19-24, wherein R² is a C8 fatty acid.

Embodiment 28: The compound according to any one of embodiments 19-22, wherein R² is a C10 fatty acid.

Embodiment 29: The compound according to any one of embodiments 19-22, wherein R² is a C12 fatty acid.

Embodiment 30: The compound of embodiment 19, wherein said compound is a compound according to the formula:

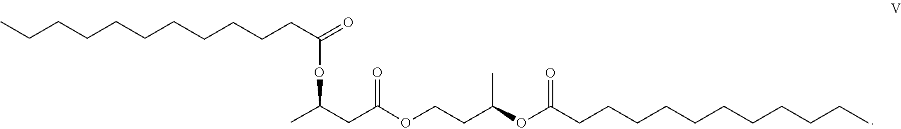 [SMILES: CCCCCCCCCCCCC(=O)O[C@H](C)CC(=O)OCC[C@@H](C)OC(=O)CCCCCCCCCCC]

Embodiment 31: The compound of embodiment 1, wherein said compound comprises a compound according to Formula II:

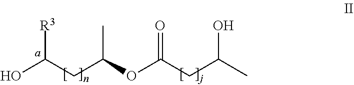

wherein at least one of n or j is 1.

Embodiment 32: The compound of embodiment 31, wherein n is 1, and j is 1, and said compound comprises a compound according to the formula:

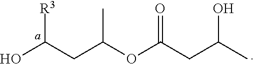

Embodiment 33: The compound of embodiment 31, wherein when n is 0, j is 1 and when j is 0, n is 1.

Embodiment 34: The compound of embodiment 33, wherein n is 0, and j is 1, and said compound comprises a compound according to the formula:

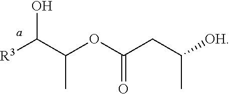

Embodiment 35: The compound of embodiment 34, wherein R³ is H.

Embodiment 36: The compound of embodiment 34, wherein R³ is CH₃.

Embodiment 37: The compound of embodiment 34, wherein said compound comprises a compound according to the formula:

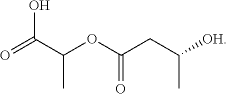

Embodiment 38: The compound of embodiment 33, wherein n is 1, and j is 0 and said compound comprise a compound according to the formula:

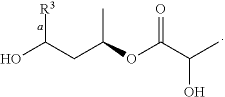

Embodiment 39: The compound of embodiment 38, wherein said compound comprises a compound according to the formula:

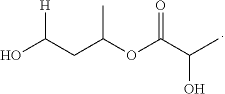

Embodiment 40: The compound of embodiment 38, wherein said compound comprises a compound according to the formula:

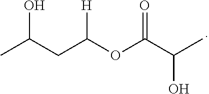

Embodiment 41: The compound of embodiment 38, wherein said compound comprises a compound according to the formula:

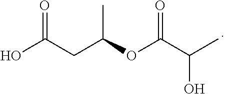

Embodiment 42: A compound according to Formula III, Formula IV, Formula V, or Formula VI:

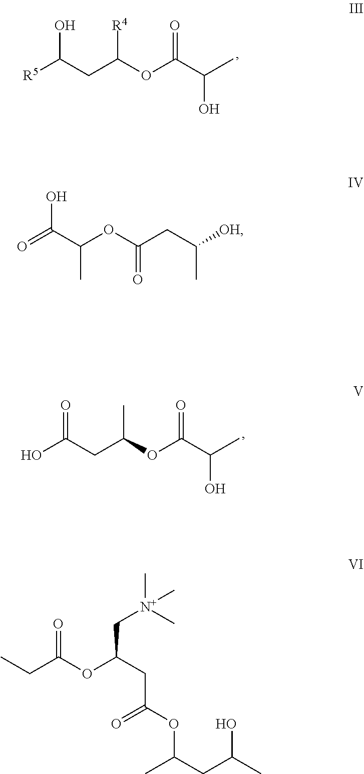

wherein: R⁴ is H or CH₃; R⁵ is H or CH₃; and when R⁴ is H, R⁵ is CH₃ and when R⁴ is CH₃, R⁵ is H.

Embodiment 43: The compound of embodiment 42, wherein said compound comprises a compound according to the formula:

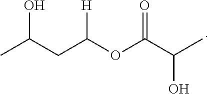

Embodiment 44: The compound of embodiment 42, wherein said compound comprises a compound according to the formula:

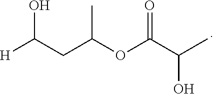

Embodiment 45: The compound of embodiment 42, wherein said compound comprises a compound according to the formula:

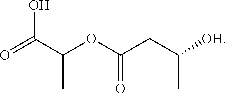

Embodiment 46: The compound of embodiment 42, wherein said compound comprises a compound according to the formula:

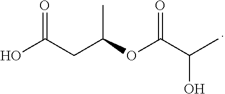

Embodiment 47: The compound of embodiment 42, wherein said compound comprises a compound according to the formula:

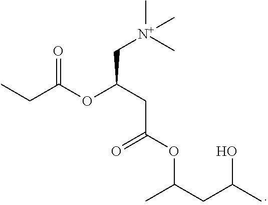 [SMILES: CCC(=O)O[C@H](CC(=O)OC(C)CC(C)O)C[N+](C)(C)C]

Embodiment 48: A pharmaceutical formulation comprising a compound according to any one of embodiments 1-47, and a pharmaceutically acceptable carrier.

Embodiment 49: The formulation of embodiment 48, wherein said formulation is for administration via a modality selected from the group consisting of intraperitoneal administration, topical administration, oral administration, inhalation administration, transdermal administration, subdermal depot administration, and rectal administration.

Embodiment 50: The formulation according to any one of embodiments 48-49, wherein said formulation is substantially sterile.

Embodiment 51: The formulation according to any one of embodiments 48-50, wherein said formulation meets FDA manufacturing guidelines for orally administered pharmaceuticals.

Embodiment 52: The formulation according to any one of embodiments 48-51, wherein said formulation is a unit dosage formulation.

Embodiment 53: An ingestible composition that comprises a compound according to any one of embodiments 1-47, and a dietetically or pharmaceutically acceptable carrier.

Embodiment 54: The composition of embodiment 53, wherein said composition comprises a dietetically acceptable carrier.

Embodiment 55: The composition of embodiment 54, wherein said composition comprises a food product, a beverage, a drink, a food supplement, a dietary supplement, a functional food, or a nutraceutical.

Embodiment 56: A food supplement comprising a compound according to any one of embodiments 1-47.

Embodiment 57: A composition comprising:
a compound according to any one of embodiments 1-47; and
one or more components of a ketogenic diet.

Embodiment 58: The composition of embodiment 57, wherein the compound and/or said substantially pure S-BHB enantiomer is present in the composition in an amount of from about 1% w/w to about 25% w/w.

Embodiment 59: The composition of embodiment 57, wherein the compound and/or said substantially pure S-BHB enantiomer is present in the composition in an amount of from about 5% w/w to about 15% w/w.

Embodiment 60: The composition of embodiment 57, wherein the compound and/or said substantially pure S-BHB enantiomer is present in the composition in an amount of about 10% w/w.

Embodiment 61: The composition according to any one of embodiments 57-60, wherein the ketogenic diet comprises a ratio by mass of fat to protein and carbohydrates of from about 2:1 to about 10:1.

Embodiment 62: The composition according to any one of embodiments 57-60, wherein the ketogenic diet comprises a ratio by mass of fat to protein and carbohydrates of from about 3:1 to about 6:1.

Embodiment 63: The composition according to any one of embodiments 57-60, wherein the ketogenic diet comprises a ratio by mass of fat to protein and carbohydrates of about 4:1.

Embodiment 64: A method of preventing the onset, slowing the progression of, and/or treating heart failure, said method comprising: administering to a subject in need thereof an effective amount of a compound according to any one of embodiments 1-47, and/or pharmaceutical formulation according to any one of embodiments 48-52.

Embodiment 65: The method of embodiment 64, wherein said heart failure is congestive heart failure.

Embodiment 66: The method of embodiment 65, wherein said congestive heart failure comprises congestive heart failure with preserved ejection fraction.

Embodiment 67: A method of treating dementia or other neurocognitive disorder, said method comprising:
administering to a subject in need thereof an effective amount of a compound according to any one of embodiments 1-47, and/or pharmaceutical formulation according to any one of embodiments 48-52.

Embodiment 68: The method of embodiment 67, wherein said method comprises treating mild cognitive impairment or Alzheimer's disease, and method comprises:
administering to a subject in need thereof an effective amount of a compound according to any one of embodiments 1-47, and/or pharmaceutical formulation according to any one of embodiments 48-52 in an amount sufficient to ameliorate one or more symptoms of Mild Cognitive Impairment and/or Alzheimer's disease.

Embodiment 69: A method of preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease, said method comprising:
administering to a subject in need thereof a compound according to any one of embodiments 1-47, and/or pharmaceutical formulation according to any one of embodiments 48-52 in an amount sufficient to prevent or delay the onset of a pre-Alzheimer's cognitive dysfunction, and/or to ameliorate one or more symptoms of a pre-Alzheimer's cognitive dysfunction, and/or to prevent or delay the progression of a pre-Alzheimer's cognitive dysfunction to Alzheimer's disease.

Embodiment 70: The method of embodiment 69, wherein said method is a method of preventing or delaying the transition from a cognitively asymptomatic pre-Alzheimer's condition to a pre-Alzheimer's cognitive dysfunction.

Embodiment 71: The method of embodiment 69, wherein said method is a method of preventing or delaying the onset of a pre-Alzheimer's cognitive dysfunction.

Embodiment 72: The method of embodiment 69, wherein said method comprises ameliorating one or more symptoms of a pre-Alzheimer's cognitive dysfunction.

Embodiment 73: The method of embodiment 69, wherein said method comprises preventing or delaying the progression of a pre-Alzheimer's cognitive dysfunction to Alzheimer's disease.

Embodiment 74: The method according to any one of embodiments 67-73, wherein said subject exhibits biomarker positivity of Aβ in a clinically normal human subject age 50 or older.

Embodiment 75: The method according to any one of embodiments 67-74, wherein said subject exhibits asymptomatic cerebral amyloidosis.

Embodiment 76: The method according to any one of embodiments 67-74, wherein said subject exhibits cerebral amyloidosis in combination with downstream neurodegeneration.

Embodiment 77: The method of embodiment 76, wherein said downstream neurodegeneration is determined by one or more elevated markers of neuronal injury selected from the group consisting of tau, and FDG uptake.

Embodiment 78: The method according to any one of embodiments 67-73, wherein said subject is a subject diagnosed with mild cognitive impairment.

Embodiment 79: The method according to any one of embodiments 67-78, wherein said subject shows a clinical dementia rating above zero and below about 1.5.

Embodiment 80: The method according to any one of embodiments 67-73, wherein the subject is at risk of developing Alzheimer's disease.

Embodiment 81: The method according to any one of embodiments 67-80, wherein the subject has a familial risk for having Alzheimer's disease.

Embodiment 82: The method according to any one of embodiments 67-80, wherein the subject has a familial Alzheimer's disease (FAD) mutation.

Embodiment 83: The method according to any one of embodiments 67-80, wherein the subject has the APOE E4 allele.

Embodiment 84: The method according to any one of embodiments 67-83, wherein administration of said compound delays or prevents the progression of MCI to Alzheimer's disease.

Embodiment 85: The method according to any one of embodiments 67-84, wherein said administration produces a reduction in the CSF of levels of one or more components selected from the group consisting of Aβ42, sAPPβ, total-Tau (tTau), phospho-Tau (pTau), APPneo, soluble Aβ40, pTau/Aβ42 ratio and tTau/Aβ42 ratio, and/or an increase in the CSF of levels of one or more components selected from the group consisting of Aβ42/Aβ40 ratio, Aβ42/Aβ38 ratio, sAPPα, sAPPα/sAPPβ ratio, sAPPα/Aβ40 ratio, and sAPPα/Aβ42 ratio.

Embodiment 86: The method according to any one of embodiments 67-85, wherein said administration produces an improvement in the cognitive abilities of the subject.

Embodiment 87: The method according to any one of embodiments 67-85, wherein said administration produces an improvement in, a stabilization of, or a reduction in the rate of decline of the clinical dementia rating (CDR) of the subject.

Embodiment 88: A method of reducing epileptiform activity in the brain of a subject, said method comprising:
administering to a subject an effective amount of a compound according to any one of embodiments 1-47, and/or pharmaceutical formulation according to any one of embodiments 48-52.

Embodiment 89: The method of embodiment 88, wherein said effective amount is sufficient to reduce epileptiform activity in the brain of said subject.

Embodiment 90: A method for treating, in a subject, one or more of epilepsy, Parkinson's disease, heart failure, traumatic brain injury, stroke, hemorrhagic shock, acute lung injury after fluid resuscitation, acute kidney injury, myocardial infarction, myocardial ischemia, diabetes, glioblastoma multiforme, diabetic neuropathy, prostate cancer, amyotrophic lateral sclerosis, Huntington's disease, cutaneous T cell lymphoma, multiple myeloma, peripheral T cell lymphoma, HIV, Niemann-Pick Type C disease, age-related macular degeneration, gout, atherosclerosis, rheumatoid arthritis and multiple sclerosis, said method comprising:
administering to a subject in need thereof an effective amount of a compound according to any one of embodiments 1-47, and/or pharmaceutical formulation according to any one of embodiments 48-52.

Embodiment 91: The method of embodiment 90, wherein the therapeutically effective amount is sufficient to reduce epileptiform activity in the brain of said subject.

Embodiment 92: A method of treating a condition which is caused by, exacerbated by or associated with elevated plasma levels of free fatty acids in a human or animal subject, said method comprising:
which method comprises administering to a subject in need thereof an effective amount of a compound according to any one of embodiments 1-47, and/or pharmaceutical formulation according to any one of embodiments 48-52.

Embodiment 93: A method of treating a condition wherein weight loss or weight gain is implicated, said method comprising:
administering to a subject in need thereof an effective amount of a compound according to any one of embodiments 1-47, and/or pharmaceutical formulation according to any one of embodiments 48-52.

Embodiment 94: A method of suppressing appetite, treating obesity, promoting weight loss, maintaining a healthy weight or decreasing the ratio of fat to lean muscle, said method comprising:
administering to a subject in need thereof an effective amount of a compound according to any one of embodiments 1-47, and/or pharmaceutical formulation according to any one of embodiments 48-52.

Embodiment 95: A method of preventing or treating a condition selected from cognitive dysfunction, a neurodegenerative disease or disorder, muscle impairment, fatigue and muscle fatigue, said method comprising:
administering to a subject in need thereof an effective amount of a compound according to any one of embodiments 1-47, and/or pharmaceutical formulation according to any one of embodiments 48-52.

Embodiment 96: A method of treating a subject suffering from a condition selected from diabetes, hyperthyroidism, metabolic syndrome X, or for treating a geriatric patient, said method comprising:
administering to a subject in need thereof an effective amount of a compound according to any one of embodiments 1-47, and/or pharmaceutical formulation according to any one of embodiments 48-52.

Embodiment 97: A method of treating, preventing, or reducing the effects of, neurodegeneration, free radical toxicity, hypoxic conditions or hyperglycemia, said method comprising:
administering to a subject in need thereof an effective amount of a compound according to any one of embodiments 1-41, and/or pharmaceutical formulation according to any one of embodiments 48-52.

Embodiment 98: A method of treating, preventing, or reducing the effects of, neurodegeneration, said method comprising:
administering to a subject in need thereof an effective amount of a compound according to any one of embodiments 1-47, and/or pharmaceutical formulation according to any one of embodiments 48-52.

Embodiment 99: The method according to any one of embodiments 97 or 98, wherein the neurodegeneration is caused by aging, trauma, anoxia or a neurodegenerative disease or disorder.

Embodiment 100: A method of preventing or treating a neurodegenerative disease or disorder selected from Alzheimer's disease, Parkinson's disease, amyotrophic lateral sclerosis, epilepsy, astrocytoma, glioblastoma and Huntington's chorea, said method comprising:
administering to a subject in need thereof an effective amount of a compound according to any one of embodiments 1-47, and/or pharmaceutical formulation according to any one of embodiments 48-52.

Embodiment 101: A method of promoting alertness or improving cognitive function in a subject, said method comprising:
administering to a subject in need thereof an effective amount of a compound according to any one of embodiments 1-41, and/or pharmaceutical formulation according to any one of embodiments 48-52.

Embodiment 102: The method according to any one of embodiments 67-101, wherein said subject is a human.

Embodiment 103: The method according to any one of embodiments 67-101, wherein said subject is a non-human mammal.

### Definitions

As used herein, the phrase “a subject in need thereof” refers to a subject, as described infra, that suffers or is at a risk of suffering (e.g., pre-disposed such as genetically pre-disposed) from the diseases or conditions listed herein.

The terms “subject,” “individual,” and “patient” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments, the subject may not be under the care or prescription of a physician or other health worker.

An “effective amount” refers to an amount effective, at dosages and for periods of time necessary, to achieve the desired therapeutic or prophylactic result. A “therapeutically effective amount” may vary according to factors such as the disease state, age, sex, and weight of the individual, and the ability of the pharmaceutical to elicit a desired response in the individual. A therapeutically effective amount is also one in which any toxic or detrimental effects of a treatment are substantially absent or are outweighed by the therapeutically beneficial effects. In certain embodiments the term “therapeutically effective amount” refers to an amount of an active agent or composition comprising the same that is effective to “treat” a disease or disorder in a mammal (e.g., a patient or a non-human mammal). In one embodiment, a therapeutically effective amount is an amount sufficient to improve at least one symptom associated with a pathology such as congestive heart failure, mild cognitive impairment (MCI), Alzheimer's disease (AD), epilepsy, Parkinson's disease, heart failure, traumatic brain injury, stroke, hemorrhagic shock, acute lung injury after fluid resuscitation, acute kidney injury, myocardial infarction, myocardial ischemia, diabetes, glioblastoma multiforme, diabetic neuropathy, prostate cancer, amyotrophic lateral sclerosis, Huntington's disease, cutaneous T cell lymphoma, multiple myeloma, peripheral T cell lymphoma, HIV, Niemann-Pick Type C disease, age-related macular degeneration, gout, atherosclerosis, rheumatoid arthritis multiple sclerosis, and the like. In certain embodiments, an effective amount is an amount sufficient to prevent advancement or the disease, delay progression, or to cause regression of a disease, or which is capable of reducing symptoms caused by the disease,

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount is less than the therapeutically effective amount.

The terms “treatment,” “treating,” or “treat” as used herein, refer to actions that produce a desirable effect on the symptoms or pathology of a disease or condition, particularly those that can be effected utilizing the compositions described herein, and may include, but are not limited to, even minimal changes or improvements in one or more measurable markers of the disease or condition being treated. Treatments also refers to delaying the onset of, retarding or reversing the progress of, reducing the severity of, or alleviating or preventing either the disease or condition to which the term applies, or one or more symptoms of such disease or condition. “Treatment,” “treating,” or “treat” does not necessarily indicate complete eradication or cure of the disease or condition, or associated symptoms thereof. In one embodiment, treatment comprises improvement of at least one symptom of a disease being treated. The improvement may be partial or complete. The subject receiving this treatment is any subject in need thereof. Exemplary markers of clinical improvement will be apparent to persons skilled in the art.

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

As used herein, the phrases “improve at least one symptom” or “improve one or more symptoms” or equivalents thereof, refer to the reduction, elimination, or prevention of one or more symptoms of pathology or disease.

The term “active agent” refers a chemical substance or compound that exerts a pharmacological action and is capable of treating, preventing or ameliorating one or more conditions/maladies (e.g., Alzheimer's disease) as described herein. Examples of active agents of interest include the compounds described herein, in particular compounds the administration of which results in an increase in ketone bodies in a subject.

As used herein, the term “prevent” or “prevention” means no infection, disorder, or pathological development if none had occurred, or no further disorder or pathological development if there had already been development of the disorder or infection. Also considered is the ability of one to prevent or reduce some or all of the symptoms associated with the disorder or disease.

As used herein, the term “pharmaceutically acceptable” refers to a material, such as a carrier or diluent, which does not abrogate the biological activity or properties of the exogenous ketone or other therapeutic agent administered as a therapeutic or prophylactic, and is relatively non-toxic, i.e., the material may be administered to an individual without causing undesirable biological effects or interacting in a deleterious manner with any of exogenous ketones or end metabolic products and other active components of the composition in which it is contained.

As used herein, the term “pharmaceutically acceptable salt” refers to derivatives of the disclosed compounds wherein the parent compound is modified by converting an existing acid or base moiety to its salt form. Nonlimiting examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids.

As used herein, the term “composition” refers to the compounds described herein combined with any other agent(s) (e.g., buffers, excipients, foods, food products, vitamins, etc.) in any usable form.

The term “pharmaceutical composition”, or “formulation” refers to at least one compound described herein and/or salt thereof, according to the present disclosure in a pharmaceutically acceptable carrier and may encompass another or other therapeutic agents.

An “oral dosage form” includes a unit dosage form prescribed or intended for oral administration.

A “food product” as used herein is an edible material composed primarily of one or more of the macronutrients protein, carbohydrate and fat, which is used in the body of an organism (e.g., a mammal) to sustain growth, repair damage, aid vital processes or furnish energy. A food product may also contain one or more micronutrients such as vitamins or minerals, or additional dietary ingredients such as flavorants and colorants.

A “functional food” is a food that is marketed as providing a health benefit beyond that of supplying pure nutrition to the consumer. A functional food typically incorporates an ingredient such as a micronutrient as mentioned above, that confers a specific medical or physiological benefit other than a nutritional effect and may carry a health claim on the packaging.

A “nutraceutical” is a food ingredient, food supplement or food product that is considered to provide a medical or health benefit, including the prevention and/or treatment of disease. In general, a nutraceutical is specifically adapted to confer a particular health benefit on the consumer, and may contain a micronutrient such as a vitamin, mineral, herb, and/or phytochemical at a higher level than would be found in a corresponding regular (natural) food product. That level is typically selected to optimize the intended health benefit of the nutraceutical when taken either as a single serving or as part of a diet regimen or course of nutritional therapy.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. In an embodiment, C₁-C₆ alkyl groups are provided herein. Nonlimiting examples include methyl, ethyl, propyl, isopropyl, butyl, isobutyl, tert butyl, pentyl, neopentyl, and hexyl. Other nonlimiting examples of C₁-C₆ alkyl include ethyl, methyl, isopropyl, isobutyl, n-pentyl, and n-hexyl.

In the term “C1-Cn fatty acid, the number (n) refers to the number of carbons comprising the primary backbone of the fatty acid including the carbon of the terminal carboxyl group. Thus, for example, hexanoic acid is a C6 fatty acid, octanoic acid is a C8 fatty acid, decanoic acid is a C10 fatty acid, and so forth.

When a carbon in a backbone is shown bracketed with a numeral (e.g., “n”), for example as

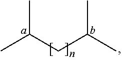

the numeral “n” indicates the number of carbons in the backbone within the bracket. When n is 0, a single bond joins carbon “a” and “b” and there are no intervening backbone carbon atoms. When n is 1 there is a single backbone carbon between carbon “a” and “b”.

As used herein, the term “substituted” means that an atom or group of atoms has replaced hydrogen as the substituent attached to another group.

A “saturated fatty acid” is a type of fatty acid in which the fatty acid chains have all single bonds.

The term “ketosis” refers to a metabolic state in a mammal characterized by elevated levels of ketone bodies in the blood or urine.

### BRIEF DESCRIPTION OF THE DRAWINGS

FIG. 1, panels A-D, illustrates various compounds that increase ketone bodies in a mammal. Panel A) Esters of BHB coupled to one or more fatty acids. Panel B) Lactate esters of BD; Panel C) Lactate esters of BHB; Panel D) One illustrative propinoyl carnitine ester of BHB.

### DETAILED DESCRIPTION

The present disclosure provides compounds, therapeutic compositions, dietary supplements, and food substances capable of inducing ketosis and/or otherwise elevating levels of ketone bodies (e.g., by provision of exogenous ketones) in a mammal. In particular, the compounds and therapeutic compositions, dietary supplements, and food substances comprising the compounds are capable of providing elevated levels of ketone bodies in the blood. Ketones, or ketone bodies, are any of the three compounds acetoacetic acid, acetone, and beta-hydroxybutyric acid which are normal intermediates in lipid metabolism. Endogenous ketone bodies have roles in energy metabolism, inflammation, and stress resistance. Their pleotropic activities at the interface of aging, metabolism, and inflammation are useful in mitigating aspects of various viral infections and resulting pathologies, particularly among patients most susceptible to severe disease.

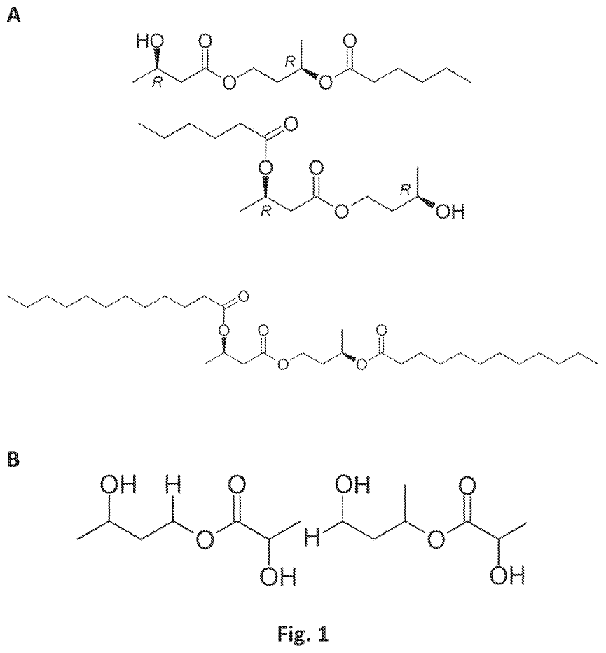

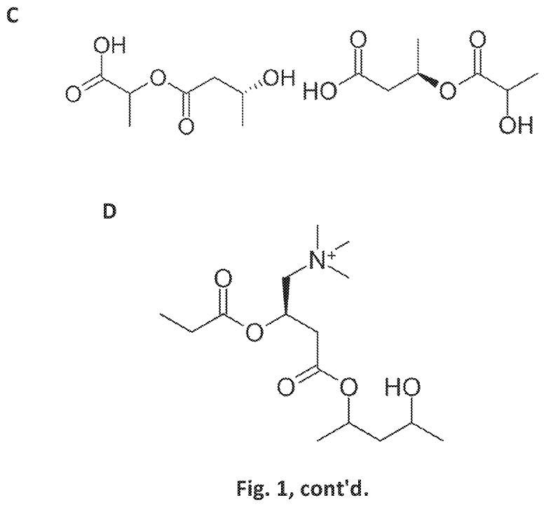

In certain embodiments the compounds provided herein include but are not limited to betahydroxybutyrate (BHB) esters and/or butanediol (BD) esters coupled to a fatty acid at one or both termini (see, e.g., FIG. 1, panel A). Without being bound to a particular theory the rationale for these molecules is the combination of a rapid release ketone ester (which does not use “classical” ketogenic pathways) with a fatty acid to also activate classical ketogenesis. In certain embodiments the compounds provided herein include but are not limited to butanediol (BD) and betahydroxybutyrate (BHD) lactate esters (see, e.g., FIG. 1, panels B & C, respectively). Without being bound to a particular theory, it is believed the combination of BHB with lactate effectively provides lactate as an additional substrate for the brain and heart. Lactate and BHB synergistically act through energy provision pathways. Additionally in certain embodiment the compounds provided herein comprise propinoyl carnitine esters of BHB (see, e.g., FIG. 1, panel D). Without being bound by a particular theory, it is believed these compounds provide improved function for cardiac energetics and find particular utility in the treatment or prophylaxis of congestive heart failure, particularly of congestive heart failure with preserved ejection fraction.

In certain embodiments it is believe the compounds described find utility in the treatment and/or prophylaxis of a number of pathologies, and are also useful as health-promoting diet supplements. The compounds described herein are hydrolyzed to produce 1) ketone bodies or immediate precursors to ketone bodies; 2) lactate or immediate precursors to lactate; and/or 3) Propinoyl 1-carnitine. These three classes of metabolites are known to positively influence function of the heart. For example, BHB and lactate work through similar energy provision pathways. Propinoyl L carnitine has anaplerotic, peripheral dilation and positive inotropic effects and can increase fatty acid oxidation and glucose utilization. BHB has additional signaling functions that may impact heart failure (e.g., impact on NLRP3 inflammasome, impact on systemic vascular resistance, and the like).

With respect to hear failure, it is noted that during rest or exercise, lactate is a major fuel for the healthy heart. In fact, as a cardiac fuel, L-lactate is preferred over glucose and free fatty acids. Accordingly, it is believed the lactate esters (in addition to the other compounds described herein) can provide exogenous lactate to improve cardiac function.

Thus, in various embodiments, the compounds, therapeutic compositions, dietary supplements, and food substances described herein find utility in the treatment and/or prophylaxis of heart disease (e.g., congestive heart failure and/or congestive heart failure with preserved ejection fraction.

In various embodiments methods and compositions described herein are useful in methods for inducing weight loss in a mammal. In certain embodiments weight loss is induced in a mammal by administering to the mammal an effective amount of one or compounds described herein.

In various embodiments compounds described herein can be administered to a mammal to alter the gut microbiome with concomitant effects on immune cells in the gut. Without being bound to a particular theory, it is believed that administration of the compounds described herein can modulate the microbiome in the gut (e.g., as illustrated by a reduction in intestinal Bifidobacterium) and can be associated concomitant downstream beneficial effects (e.g., a reduction in inflammation). In some instances, the amount of the one or more compounds administered to the subject is sufficient to alter the microbiome and/or to reduce intestinal inflammation in a mammal.

In various embodiments one or more of the compounds described herein can be administered to a mammal to reduce intestinal Th17 cell accumulation and/or inflammation in the intestine of a mammal. In some instances, the amount of the one or more compounds administered to the subject is sufficient reduce intestinal Th17 cell accumulation and/or inflammation in the intestine of a mammal.

The compounds described herein (see, e.g., FIG. 1) are believed to be effective to reduce plasma levels of fatty acids. Accordingly, in certain embodiments, it is believed that these compounds, and formulations, therapeutic compositions, dietary supplements, and food substances comprising these compounds can be used to reduce the level of free fatty acids circulating in the plasma of a subject (e.g., a human, or a non-human mammal). As such they may be used to treat a condition that is caused by, exacerbated by or associated with elevated plasma levels of free fatty acids in a human or non-human animal subject. Thus, in certain embodiments, a human or animal subject may be treated by a method that comprises the administration thereto of one or more compounds described herein and/or formulations, therapeutic compositions, dietary supplements, and food substances comprising these compounds. The condition of the subject may thereby be improved or ameliorated.

Conditions that are caused by, exacerbated by or associated with elevated plasma levels of free fatty acids include, but are not limited to, neurodegenerative diseases or disorders, for instance Alzheimer's disease, Parkinson's disease, Huntington's chorea; hypoxic states, for instance angina pectoris, extreme physical exertion, intermittent claudication, hypoxia, stroke and myocardial infarction; insulin resistant states, for instance infection, stress, obesity, diabetes and heart failure; and inflammatory states including infection and autoimmune disease.

In addition to reducing plasma levels of fatty acids, it is believed that the compounds described herein act on the appetite centers in the brain. In particular, it is believed these enantiomers can increase the levels of various anorexigenic neuropeptides (neuropeptides known to be associated with decreased food intake and decreased appetite) in the appetite centers of the brain and also induce higher levels of malonyl CoA, a metabolite associated with decreased appetite and food intake.

Accordingly, in certain embodiments, the compounds described herein and/or formulations, therapeutic compositions, dietary supplements, and food substances comprising these compounds can be useful in treating a condition wherein weight loss or weight gain is implicated. For example, the enantiomers and/or compositions/formulations thereof may be used in suppressing appetite, treating obesity, promoting weight loss, maintaining a healthy weight or decreasing the ratio of fat to lean muscle in a subject. In various embodiments the subject in each case may be a healthy subject or a compromised subject. A healthy subject may be, for instance, an individual of healthy weight for whom physical performance and/or physical appearance is important. Examples include, but are not limited to, members of the military, athletes, body builders and fashion models. A compromised subject may be an individual of non-healthy weight, for instance an individual who is overweight, clinically obese or clinically very obese. A compromised subject may alternatively be an individual of healthy or non-healthy weight who is suffering from a clinical condition, for instance a condition listed below.

An individual of healthy weight typically has a body mass index (BMI) of about 18.5 to about 24.9, an individual who is overweight typically has a body mass index (BMI) of from about 25 to about 29.9, an individual who is clinically obese typically has a body mass index of from about 30 to 39.9, and an individual who is clinically very obese typically has a body mass index of about 40 or more.

In addition to reducing plasma levels of fatty acids and acting on the appetite centers in the brain, it is believed the compounds described herein and/or formulations, therapeutic compositions, dietary supplements, and food substances comprising these compounds can increase brain metabolic efficiency, by increasing brain phosphorylation potential and the ΔG′ of ATP hydrolysis. Accordingly, it is believed the compounds described herein and/or formulations, therapeutic compositions, dietary supplements, and food substances comprising these compounds can promote improved cognitive function and can be used to treat cognitive dysfunction or to reduce the effects of neurodegeneration.

It is also believed the compounds described herein and/or formulations, therapeutic compositions, dietary supplements, and food substances comprising these compounds can increase the level of the neuropeptide Brain Derived Neurotropic Factor (BDNF) in both the paraventricular nucleus (the appetite center of the brain) and the hippocampus (a part of the brain known to be important for memory). BDNF is known to prevent apoptosis and promote neuronal growth in basal ganglia and other areas of interest, thus the increased levels of BDNF produced by the enantiomers described herein and/or compositions/formulations thereof are expected to inhibit neurodegeneration, limit neural tissue death after hypoxia or trauma and promote neural tissue growth.

The compounds described herein and/or formulations, therapeutic compositions, dietary supplements, and food substances comprising these compounds are also believed to increase the level of the anorexigenic neuropeptide Cocaine-and-Amphetamine Responsive Transcript (CART). CART is known to promote alertness as well as to decrease appetite. Thus, the increased levels of CART produced by the compounds described herein and/or formulations, therapeutic compositions, dietary supplements, and food substances comprising these compounds are expected to improve cognitive function. The compounds described herein and/or formulations, therapeutic compositions, dietary supplements, and food substances comprising these compounds are therefore expected to be useful for (a) promoting alertness and improved cognitive function; and/or (b) inhibiting neurodegeneration.

In certain embodiments the compounds described herein and/or formulations, therapeutic compositions, dietary supplements, and food substances comprising these compounds are provided for use in promoting alertness or improving cognitive function, or in treating cognitive dysfunction.

In certain embodiments the compounds described herein and/or formulations, therapeutic compositions, dietary supplements, and food substances comprising these compounds are provided for use in treating, preventing, or reducing the effects of, neurodegeneration, free radical toxicity, hypoxic conditions, or hyperglycemia.

In one illustrative but non-limiting embodiment, the compounds described herein and/or formulations, therapeutic compositions, dietary supplements, and food substances comprising these compounds are provided for use in in treating, preventing, or reducing the effects of, neurodegeneration. Thus, it is believed the compounds described herein and/or formulations, therapeutic compositions, dietary supplements, and food substances comprising these compounds can be used to treat, prevent, or reduce the effects of neurodegeneration arising from any particular cause. The neurodegeneration may for instance be caused by a neurodegenerative disease or disorder, or may be caused by aging, trauma, anoxia and the like. Examples of neurodegenerative diseases or disorders that can be treated using S-enantiomers described herein and/or compositions/formulations thereof include, but are not limited to Alzheimer's disease, Parkinson's disease, amyotrophic lateral sclerosis, epilepsy, astrocytoma, glioblastoma, and Huntington's chorea.

Further examples of conditions which the compounds described herein and/or formulations, therapeutic compositions, dietary supplements, and food substances comprising these compounds may be used to prevent or treat include, but are not limited to muscle impairment, fatigue and muscle fatigue. Muscle impairment and muscle fatigue may be prevented or treated in a healthy or compromised subject. A compromised subject may be, for instance, an individual suffering from fibromyalgia, or from myalgic encephalomyelitis (ME, or chronic fatigue syndrome), or the symptoms thereof. In certain embodiments the compounds described herein and/or formulations, therapeutic compositions, dietary supplements, and food substances comprising these compounds may be used to treat a subject suffering from a condition such as diabetes, metabolic syndrome X or hyperthyroidism, or a geriatric patient.

In certain embodiments methods of mild cognitive impairment (MCI) or Alzheimer's disease are provided wherein the methods involve administering to a subject in need thereof one or more of the compounds described herein and/or formulations, therapeutic compositions, dietary supplements, and food substances comprising these compounds in an amount sufficient to ameliorate one or more symptoms of Mild Cognitive Impairment and/or Alzheimer's disease. Similarly methods are also provided for preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease, wherein the methods involve administering to a subject in need thereof one or more of the compounds described herein and/or formulations, therapeutic compositions, dietary supplements, and food substances comprising these compounds in an amount sufficient to prevent or delay the onset of a pre-Alzheimer's cognitive dysfunction, and/or to ameliorate one or more symptoms of a pre-Alzheimer's cognitive dysfunction, and/or to prevent or delay the progression of a pre-Alzheimer's cognitive dysfunction to Alzheimer's disease. In certain embodiments the method is a method of preventing or delaying the transition from a cognitively asymptomatic pre-Alzheimer's condition to a pre-Alzheimer's cognitive dysfunction. In certain embodiments the method is a method of preventing or delaying the onset of a pre-Alzheimer's cognitive dysfunction. In certain embodiments the method comprises ameliorating one or more symptoms of a pre-Alzheimer's cognitive dysfunction. In certain embodiments the method comprises preventing or delaying the progression of a pre-Alzheimer's cognitive dysfunction to Alzheimer's disease. In certain embodiments the subject is one that exhibits biomarker positivity of Aβ in a clinically normal subject (e.g., a human subject age 50 or older). In certain embodiments the subject exhibits asymptomatic cerebral amyloidosis. In certain embodiments the subject exhibits cerebral amyloidosis in combination with downstream neurodegeneration (e.g., as determined by one or more elevated markers of neuronal injury selected from the group consisting of tau, and FDG uptake). In certain embodiments the subject is a subject diagnosed with mild cognitive impairment. In certain embodiments the subject shows a clinical dementia rating above zero and below about 1.5. In certain embodiments the subject is at risk of developing Alzheimer's disease (e.g., the subject has a familial risk for having Alzheimer's disease (e.g., the FAD mutation, the APOE E4 allele). In certain embodiments the administration of the compound delays or prevents the progression of MCI to Alzheimer's disease. In certain embodiments the administration produces a reduction in the CSF of levels of one or more components selected from the group consisting of Aβ42, sAPPβ, total-Tau (tTau), phospho-Tau (pTau), APPneo, soluble Aβ40, pTau/Aβ42 ratio and tTau/Aβ42 ratio, and/or an increase in the CSF of levels of one or more components selected from the group consisting of Aβ42/Aβ40 ratio, Aβ42/Aβ38 ratio, sAPPα, sAPPα/sAPPβ ratio, sAPPα/Aβ40 ratio, and sAPPα/Aβ42 ratio and/or produces an improvement in the cognitive abilities of the subject, and/or an improvement in, a stabilization of, or a reduction in the rate of decline of the clinical dementia rating (CDR) of the subject.

In certain embodiments the compounds described herein and/or formulations, therapeutic compositions, dietary supplements, and food substances comprising these compounds are administered to a subject to increase cognition in the subject. For example, the subject methods may include administering an amount of the compounds described herein and/or formulations, therapeutic compositions, dietary supplements, and food substances comprising these compounds to increase cognition in the subject by 5% or more, such as 10% or more, such as 15% or more, such as 25% or more, such as 40% or more, such as 50% or more, such as 75% or more, such as 90% or more, such as 95% or more, such as 99% or more and including increasing cognition in the subject by 99.9% or more.

In yet other instances, the amount of the one or more t the compounds described herein and/or formulations, therapeutic compositions, dietary supplements, and food substances comprising these compounds administered to the subject is sufficient to reduce the rate of decline of cognition in the subject. For example, the subject methods may include administering an amount of the S-enantiomers described herein and/or and compositions/formulations thereof to decrease the rate of decline of cognition in the subject by 5% or more, such as 10% or more, such as 15% or more, such as 25% or more, such as 40% or more, such as 50% or more, such as 75% or more, such as 90% or more, such as 95% or more, such as 99% or more and including reducing the rate of decline in cognition in the subject by 99.9% or more.

Cognition level in a subject may be assessed by any convenient protocol, including but not limited to the Montreal Cognitive Assessment (MoCA), St. Louis University Mental State Exam (SLUMS), Mini Mental State Exam (MMSE), and, for research purposes, Alzheimer's Disease Assessment Scale, Cognition (ADAS-Cog), as well as assessments including Activities of Daily Living (ADLs) and Instrumental Activities of Daily Living (IADLs).

In certain embodiments, one or more of the compounds described herein and/or formulations, therapeutic compositions, dietary supplements, and food substances comprising these compounds are administered to improve a subject's daily function such as determined by assessments by Activities of Daily Living (ADLs) and Instrumental Activities of Daily Living (IADLs).

In other embodiments, one or more of the compounds described herein and/or formulations, therapeutic compositions, dietary supplements, and food substances comprising these compounds are administered to reduce agitated behaviors in the subject. For example, the subject methods may include administering an amount of one or more of the S-enantiomer(s) described herein and/or and compositions/formulations thereof sufficient to reduce agitated behaviors in the subject by 5% or more, such as 10% or more, such as 15% or more, such as 25% or more, such as 40% or more, such as 50% or more, such as 75% or more, such as 90% or more, such as 95% or more, such as 99% or more and including reducing agitated behaviors in the subject in the subject by 99.9% or more. Agitated behavior may be assessed by any convenient protocol such as assessed by the Neuropsychiatric Inventory (NPI).

In yet other embodiments, one or more of the compounds described herein and/or formulations, therapeutic compositions, dietary supplements, and food substances comprising these compounds are administered to reduce delirium in the subject. For example, the subject methods may include administering an amount of one or more of the S-enantiomer(s) described herein and/or and compositions/formulations thereof sufficient to reduce delirium in the subject by 5% or more, such as 10% or more, such as 15% or more, such as 25% or more, such as 40% or more, such as 50% or more, such as 75% or more, such as 90% or more, such as 95% or more, such as 99% or more and including reducing delirium in the subject in the subject by 99.9% or more.

In still other embodiments, one or more of the compounds described herein and/or formulations, therapeutic compositions, dietary supplements, and food substances comprising these compounds are administered to the subject to reduce stress experienced by a caregiver to the subject. For example, the subject methods may include administering an amount of one or more of the compounds described herein and/or formulations, therapeutic compositions, dietary supplements, and food substances comprising these compounds sufficient to reduce stress experienced by a caregiver to the subject by 5% or more, such as 10% or more, such as 15% or more, such as 25% or more, such as 40% or more, such as 50% or more, such as 75% or more, such as 90% or more, such as 95% or more, such as 99% or more and including reducing stress experienced by a caregiver to the subject in the subject by 99.9% or more. Caregiver stress may be assessed by any convenient protocol such as assessed by the Perceived Stress Scale (PSS).

Also provided are methods for treating one or more of epilepsy, Parkinson's disease, heart failure, traumatic brain injury, stroke, hemorrhagic shock, acute lung injury after fluid resuscitation, acute kidney injury, myocardial infarction, myocardial ischemia, diabetes, glioblastoma multiforme, diabetic neuropathy, prostate cancer, amyotrophic lateral sclerosis, Huntington's disease, cutaneous T cell lymphoma, multiple myeloma, peripheral T cell lymphoma, HIV, Niemann-Pick Type C disease, age-related macular degeneration, gout, atherosclerosis, rheumatoid arthritis and multiple sclerosis by administering one or more of the compounds described herein and/or formulations, therapeutic compositions, dietary supplements, and food substances comprising these compounds.

The aforementioned conditions are examples of conditions that may be caused by, exacerbated by or associated with elevated plasma levels of free fatty acids accordingly, it is believed the compounds described herein and/or formulations, therapeutic compositions, dietary supplements, and food substances comprising these compounds may be used to treat these conditions.

However, in other embodiments, it is believed the compounds described herein and/or formulations, therapeutic compositions, dietary supplements, and food substances comprising these compounds may be used to treat a subject suffering from a condition such as diabetes, hyperpyrexia, hyperthyroidism, metabolic syndrome X, fever, and/or an infection.

In certain embodiments the compounds described herein and/or formulations, therapeutic compositions, dietary supplements, and food substances comprising these compounds can be administered in combination with one or more additional agents. Such agents include but are not limited to micronutrients and medicaments. In certain embodiments the S-enantiomer(s) and the additional agent(s) may be formulated together in a single composition for ingestion. Alternatively, in certain embodiments, the compounds described herein and/or formulations, therapeutic compositions, dietary supplements, and food substances comprising these compounds and the additional agent may be formulated separately for separate, simultaneous or sequential administration.

When the additional agent is a medicament it may be, for instance, a standard therapy for a condition from which the subject is suffering. For instance, in certain embodiments, the compounds described herein and/or formulations, therapeutic compositions, dietary supplements, and food substances comprising these compounds can be administered in combination with conventional anti-diabetic agents to a subject suffering from diabetes. Conventional anti-diabetic agents include, but are not limited to, insulin sensitizers such as the thiazolidinediones, insulin secretogogues such as sulphonylureas, biguanide antihyperglycemic agents such as metformin, and combinations thereof.

In certain embodiments, when the additional agent comprises a micronutrient, it may be, for instance, a mineral or a vitamin. Examples include, but are not limited to, iron, calcium, magnesium, vitamin A, the B vitamins, vitamin C, vitamin D and vitamin E.

Ketone bodies act on niacin receptors. Accordingly, in certain embodiments, the compounds described herein and/or formulations, therapeutic compositions, dietary supplements, and food substances comprising these compounds may advantageously be administered in combination with niacin (vitamin B3) as both ketone bodies and niacin act on adipose tissue to inhibit free fatty acid release.

In certain embodiments the compounds described herein and/or formulations, therapeutic compositions comprising these compounds can be formulated into an ingestible composition that further comprises a dietetically or pharmaceutically acceptable carrier. The compositions may include, but are not limited to, food products, beverages, drinks, supplements, dietary supplements, functional foods, nutraceuticals or medicaments.

In various embodiments the concentration of the compounds described herein in the ingestible composition depends on a variety of factors, including the particular format of the composition, the intended use of the composition and the target population. Generally, the composition will contain the compound(s) in an amount effective to reduce plasma levels of free fatty acids. Typically, the amount is that required to achieve a circulating concentration of the compound described herein ranges from about 10 μM to about 20 mM, or from about 50 μM to about 10 mM, or from about 100 μM to about 5 mM, in a subject (e.g., a human or non-human mammal) that ingests the composition. In one embodiment, an amount is used to achieve a circulating concentration of from about 0.7 mM to about 5 mM, for example from about 1 mM to about 5 mM.

### Active Agent(s)—Ketone Ester(s).

In certain embodiments the active agent(s) compounds provided herein comprises betahydroxybutyrate (BHB) esters and/or butanediol (BD) esters coupled to a fatty acid at one or both termini (see, e.g., FIG. 1, panel A). Accordingly, in certain embodiments, a compound according to Formula I or Formula II is provided:

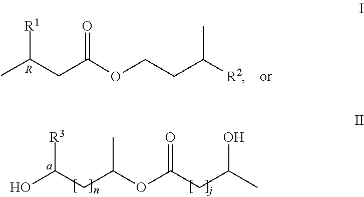

wherein R¹ is OH or C(4-14) saturated fatty acid; R² is OH or C(4-14) saturated fatty acid; at least one of R¹ or R² is said saturated fatty acid; R³ is H, CH₃, or O; n and j are 0 or 1, and when n is 0, j is 1 and when n is 1, j is 0; and when R³ is O, bond “a” is a double bond.

In certain embodiments the compound comprises a compound according to Formula I:

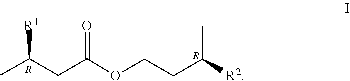

In certain embodiments R¹ is OH. In certain embodiments of Formula I, R² is a C(4-10) fatty acid, or a C(4-6) fatty acid. In certain embodiments R² is a C6 fatty acid. In certain embodiments R² is a C8 fatty acid. In certain embodiments R² is a C10 fatty acid. In certain embodiments R² is a C12 fatty acid.

In certain embodiments the compound is a compound according to the formula:

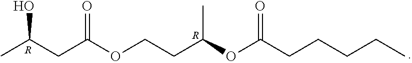

In certain embodiments of the foregoing embodiment, R² is OH. In certain of the foregoing embodiments R¹ is a C(4-10) fatty acid or a C(4-6) fatty acid. In certain embodiments R¹ is a C6 fatty acid. In certain embodiments R¹ is a C8 fatty acid. In certain embodiments R¹ is a C10 fatty acid. In certain embodiments R¹ is a C12 fatty acid.

In certain embodiments the compound is a compound according to the formula:

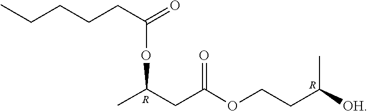

In certain of the foregoing embodiments R¹ is a C(4-14) saturated fatty acid; and R² is a C(4-14) saturated fatty acid. In certain embodiments R¹ is a C4 saturated fatty acid. In certain embodiments R¹ is a C6 saturated fatty acid. In certain embodiments R¹ is a C8 saturated fatty acid. In certain embodiments R¹ is a C10 saturated fatty acid. In certain embodiments R¹ is a C12 saturated fatty acid. In certain embodiments of these embodiments R² is a C4 fatty acid, or a C6 fatty acid, or a C8 fatty acid, or a C10 fatty acid, or a C12 fatty acid. In certain embodiments the compound is a compound according to the formula:

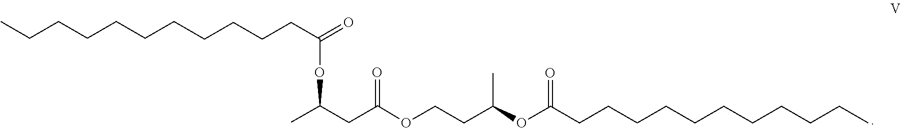 [SMILES: CCCCCCCCCCCCC(=O)O[C@H](C)CC(=O)OCC[C@@H](C)OC(=O)CCCCCCCCCCC]

In certain embodiments the compound comprises a compound according to Formula II:

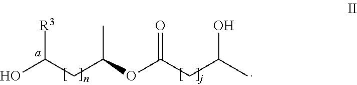

In certain embodiments n is 1, and j is 1, and said compound comprises a compound according to the formula:

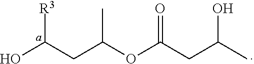

In certain embodiments R³ is H. In certain embodiments R³ is CH₃. In certain embodiments R³ is O. In certain embodiments when n is 0, j is 1 and when j is 0, n is 1.

In certain embodiments when n is 0, and j is 1, the compound comprises a compound according to the formula:

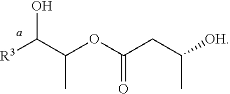

In certain embodiments R³ is H. In certain embodiments R³ is CH₃.

In certain embodiments the compound comprises a compound according to the formula:

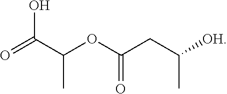

In certain embodiments when n is 1, and j is 0 and said compound comprise a compound according to the formula:

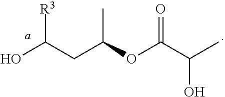

In certain embodiments the compound comprises a compound according to the formula:

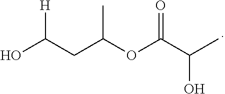

In certain embodiments the compound comprises a compound according to the formula:

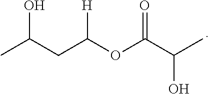

In certain embodiments the compound comprises a compound according to the formula:

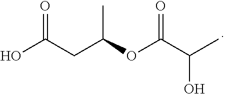

In certain embodiments the compounds provided herein include but are not limited to butanediol (BD) and betahydroxybutyrate (BHD) lactate esters (see, e.g., FIG. 1, panels B & C, respectively). Thus, for example, in certain embodiments a compound according to Formula III, Formula IV, or Formula V is provided:

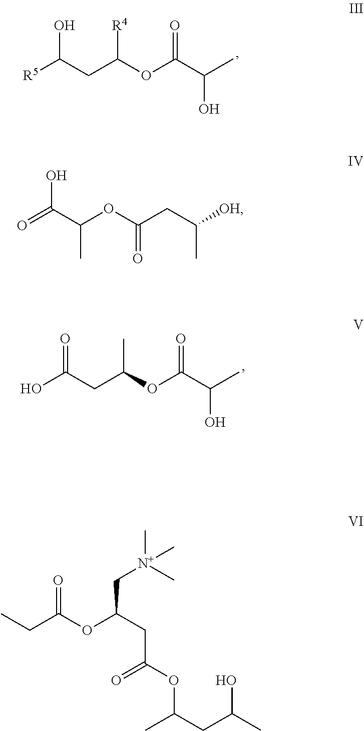

wherein R⁴ is H or CH₃; R⁵ is H or CH₃; and when R⁴ is H, R⁵ is CH₃ and when R⁴ is CH₃, R⁵ is H.

In certain embodiments the compound comprises a compound according to the formula:

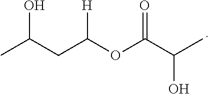

In certain embodiments the compound comprises a compound according to the formula:

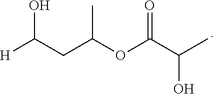

In certain embodiments the compound comprises a compound according to the formula:

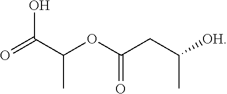

In certain embodiments the compound comprises a compound according to the formula:

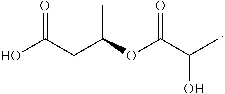

Additionally in certain embodiment the compounds provided herein comprise propinoyl carnitine esters of BHB (see, e.g., FIG. 1, panel D). In one illustrative, but non-limiting embodiment, the compound (active agent) comprises a compound according to the formula:

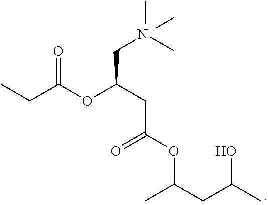 [SMILES: CCC(=O)O[C@H](CC(=O)OC(C)CC(C)O)C[N+](C)(C)C]

The foregoing compounds and formulas are illustrative and non-liming. Using the teaching provided herein numerous other esters of BHB coupled to one or more fatty acids, lactate esters of BD, lactate esters of BHB, and propinoyl carnitine esters of BHB will be available to one of skill in the art.

The compounds (e.g., ketone esters) described herein, may be prepared by chemical synthesis protocols known to those of skill in the art (See e.g., Green et al., “Protective Groups in Organic Chemistry,” (Wiley, 2nd ed. 1991); Harrison et al., “Compendium of Synthetic Organic Methods,” Vols. 1 8 (John Wiley and Sons, 1971 1996); “Beilstein Handbook of Organic Chemistry,” Beilstein Institute of Organic Chemistry, Frankfurt, Germany; Feiser et al., “Reagents for Organic Synthesis,” Volumes 1 17, (Wiley Interscience); Trost et al., “Comprehensive Organic Synthesis,” (Pergamon Press, 1991); “Theilheimer's Synthetic Methods of Organic Chemistry,” Volumes 1 45, (Karger, 1991); March, “Advanced Organic Chemistry,” (Wiley Interscience), 1991; Larock “Comprehensive Organic Transformations,” (VCH Publishers, 1989); Paquette, “Encyclopedia of Reagents for Organic Synthesis,” (John Wiley & Sons, 1995), Bodanzsky, “Principles of Peptide Synthesis,” (Springer Verlag, 1984); Bodanzsky, “Practice of Peptide Synthesis,” (Springer Verlag, 1984). Further, starting materials may be obtained from commercial sources or via well-established synthetic procedures. In this regard it is noted that BHB and BD are commercially available and esterification chemistries are routine to those of skill in the art.

In certain embodiments the synthesis of the compounds described herein can readily be contracted to a CRO, e.g., New England Discovery Partners, LLC (Branford, CT), and the like.

### Components of a Ketogenic Diet.

In various embodiments the compounds described herein are administered as components of a ketogenic diet. A ketogenic diet is a very low carbohydrate, high fat diet. Implementing a ketogenic diet involves significantly reducing carbohydrate intake and replacing it with fat. When this happens, the body becomes efficient at utilizing fat for energy production and turning fat into ketones in the liver that can supply energy for the brain.

Illustrative, but non-limiting ketogenic diets include, but are not limited to: 1) The standard ketogenic diet that is a low carbohydrate moderate protein and high fat diet typically containing about 70% fat, about 20% protein and only about 10% carbohydrates; and 2) The high protein ketogenic diet which is similar to a standard ketogenic diet, but includes more protein typically comprising a ration of about 60% fat, about 35% protein, and about 5% carbohydrates. Exemplary ketogenic diets and components thereof are described for example in U.S. Pat. No. 6,207,856, the disclosure of which is incorporated by reference herein.

It will be recognized that the foregoing ketogenic diets are illustrative and non-limiting and using the teachings provided herein the compounds described herein can readily be used as components in a wide number of ketogenic diets.

Pharmaceutical and/or Dietary Formulations.

In certain embodiments the compounds described herein may be formulated into a medicament or a dietary supplement by mixing with a dietetically or pharmaceutically acceptable carrier or excipient. In various embodiments, such a carrier or excipient may comprise, but is not limited to, a solvent, dispersion medium, coating, isotonic or absorption delaying agent, sweetener or the like. These include any and all solvents, dispersion media, coatings, isotonic and absorption delaying agents, sweeteners and the like. Suitable carriers may be prepared from a wide range of materials including, but not limited to, diluents, binders and adhesives, lubricants, disintegrants, coloring agents, bulking agents, flavoring agents, sweetening agents and miscellaneous materials such as buffers and adsorbents that may be needed in order to prepare a particular dosage form. The use of such media and agents for pharmaceutically active substances is well known in the art.

The compounds described herein can be administered in the “native” form or, if desired, in the form of a derivative provided the derivative is suitable pharmacologically, e.g., effective in the present method(s). Derivatives of the compounds described herein can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of pharmaceutically formulating the compounds described herein as salts, esters, free acids, amides, and the like are well known to those of skill in the art. Certain salts can include, for example, halide salts. Certain basic salts include alkali metal salts, e.g., the sodium salt, and copper salts. Illustrative anionic salt forms include, but are not limited to acetate, benzoate, benzylate, bitartrate, bromide, carbonate, chloride, citrate, edetate, edisylate, estolate, formate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the compounds identified herein are useful for parenteral, topical, oral, nasal (or otherwise inhaled), rectal, or local administration, such as by aerosol or transdermally, for prophylactic and/or therapeutic treatment of one or more of the pathologies/indications described herein (e.g., amyloidogenic pathologies).

The active agent(s) described herein can also be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the compound(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the compounds described herein, or excipients or other stabilizers and/or buffers.

Other physiologically acceptable materials, particularly of use in the preparation of tablets, capsules, gel caps, and the like include, but are not limited to binders, diluent/fillers, disintegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g., calcium carbonate, carboxymethylcellulose calcium, sodium starch glycollate, crospovidone etc.), a binder (e.g., alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the compound(s) described herein and the resulting composition is compressed. When necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) described herein and on the particular physio-chemical characteristics of the agent(s).

In certain embodiments, the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectable, implantable sustained-release formulations, mucoadherent films, topical varnishes, lipid complexes, etc.

Pharmaceutical compositions comprising the compounds described herein can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the compound(s) into preparations that can be used pharmaceutically. Proper formulation is dependent upon the route of administration chosen.

Systemic formulations include, but are not limited to, those designed for administration by injection, e.g., subcutaneous, intravenous, intramuscular, intrathecal or intraperitoneal injection, as well as those designed for transdermal, transmucosal oral or pulmonary administration. For injection, the compounds described herein can be formulated in aqueous solutions, preferably in physiologically compatible buffers such as Hanks solution, Ringer's solution, or physiological saline buffer and/or in certain emulsion formulations. The solution can contain formulatory agents such as suspending, stabilizing and/or dispersing agents. In certain embodiments, the compounds described herein can be provided in powder form for constitution with a suitable vehicle, e.g., sterile pyrogen-free water, before use. For transmucosal administration, penetrants appropriate to the barrier to be permeated can be used in the formulation. Such penetrants are generally known in the art.

For oral administration, the compounds can be readily formulated by combining the compound(s) with pharmaceutically acceptable carriers well known in the art. Such carriers enable the compounds described herein to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added. For buccal administration, the compositions may take the form of tablets, lozenges, etc. formulated in conventional manner.

For administration by inhalation, the compounds described herein are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

In various embodiments, the compounds described herein can be formulated in rectal or vaginal compositions such as suppositories or retention enemas, e.g., containing conventional suppository bases such as cocoa butter or other glycerides.

In addition to the formulations described previously, the compounds described herein may also be formulated as a depot preparation. Such long-acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that may be used to protect and deliver pharmaceutically active compounds. Certain organic solvents such as dimethylsulfoxide also can be employed, although usually at the cost of greater toxicity. Additionally, the compounds may be delivered using a sustained-release system, such as semipermeable matrices of solid polymers containing the therapeutic agent. Various uses of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

In certain embodiments, the compounds described herein (e.g., BH-BD) and/or formulations described herein are administered orally. This is readily accomplished by the use of tablets, caplets, lozenges, liquids, and the like.

In certain embodiments, the compound(s) and/or formulations described herein are administered systemically (e.g., orally, or as an injectable) in accordance with standard methods well known to those of skill in the art. In other embodiments, the agents can also be delivered through the skin using conventional transdermal drug delivery systems, e.g., transdermal “patches” wherein the compound(s) and/or formulations described herein are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the compounds and any other materials that are present.

In certain embodiments, one or more compounds described herein can be provided as a “concentrate”, e.g., in a storage container (e.g., in a premeasured volume) ready for dilution, or in a soluble capsule ready for addition to a volume of water, alcohol, hydrogen peroxide, or other diluent.

In certain embodiments, the compounds described herein are suitable for oral administration. In various embodiments, the compound(s) in the oral compositions can be either coated or non-coated. The preparation of enteric-coated particles is disclosed for example in U.S. Pat. Nos. 4,786,505 and 4,853,230.

In various embodiments, compositions contemplated herein typically comprise one or more of the compound(s) described herein in an effective amount to achieve a pharmacological effect or therapeutic improvement (e.g., induction of weight loss) without undue adverse side effects. Illustrative pharmacological effects or therapeutic improvements include, but are not limited to, a reduction or cessation in the rate of bone resorption at one or more locations, an increase in bone density, a reduction in tumor volume, a reduction in arthritic pathology, and the like.

In various embodiments, the typical daily dose of compounds described herein (e.g., BH-BD) varies and will depend on various factors such as the individual requirements of the subjects to be treated. In certain embodiments, the daily dose of compounds can be in the range of 100-1,000 mg/kg, or from about 200-700 mg/kg, or from about 300-600 mg/kg. In one illustrative embodiment a standard approximate amount of the compounds described above present in the composition can be typically about 1 to about 10 g, or from 2 to about 5 g, or from about 2 to about 4 g. In certain embodiments the compounds are administered only once, or for follow-up as required. In certain embodiments the compounds and/or formulations thereof are administered once a day, in certain embodiments, administered twice a day, in certain embodiments, administered 3 times/day, and in certain embodiments, administered 4, or 6, or 6 or 7, or 8 times/day.

In certain embodiments the active agents described herein are formulated in a single oral dosage form containing all active ingredients. Such oral formulations include solid and liquid forms. It is noted that solid formulations typically provide improved stability as compared to liquid formulations and can often afford better patient compliance.

In one illustrative embodiment, the one or more of the compounds described herein are formulated in a single solid dosage form such as single- or multi-layered tablets, suspension tablets, effervescent tablets, powder, pellets, granules or capsules comprising multiple beads as well as a capsule within a capsule or a double chambered capsule. In another embodiment, the compounds described herein may be formulated in a single liquid dosage form such as suspension containing all active ingredients or dry suspension to be reconstituted prior to use.

In certain embodiments, the compounds described herein are formulated as enteric-coated delayed-release granules or as granules coated with non-enteric time-dependent release polymers in order to avoid contact with the gastric juice. Non-limiting examples of suitable pH-dependent enteric-coated polymers include, for example, cellulose acetate phthalate, hydroxypropylmethylcellulose phthalate, polyvinylacetate phthalate, methacrylic acid copolymer, shellac, hydroxypropylmethylcellulose succinate, cellulose acetate trimellitate, and mixtures of any of the foregoing. A suitable commercially available enteric material, for example, is sold under the trademark EUDRAGIT L 100-55®. This coating can be spray coated onto a substrate.

Illustrative non-enteric-coated time-dependent release polymers include, for example, one or more polymers that swell in the stomach via the absorption of water from the gastric fluid, thereby increasing the size of the particles to create thick coating layer. The time-dependent release coating generally possesses erosion and/or diffusion properties that are independent of the pH of the external aqueous medium. Thus, the active ingredient is slowly released from the particles by diffusion or following slow erosion of the particles in the stomach.

Illustrative non-enteric time-dependent release coatings are for example: film-forming compounds such as cellulosic derivatives, such as methylcellulose, hydroxypropyl methylcellulose (HPMC), hydroxyethylcellulose, and/or acrylic polymers including the non-enteric forms of the EUDRAGIT® brand polymers. Other film-forming materials can be used alone or in combination with each other or with the ones listed above. These other film forming materials generally include, for example, poly(vinylpyrrolidone), Zein, poly(ethylene glycol), poly(ethylene oxide), poly(vinyl alcohol), poly(vinyl acetate), and ethyl cellulose, as well as other pharmaceutically acceptable hydrophilic and hydrophobic film-forming materials. These film-forming materials may be applied to the substrate cores using water as the vehicle or, alternatively, a solvent system. Hydro-alcoholic systems may also be employed to serve as a vehicle for film formation.

Other materials suitable for making the time-dependent release coating of the compounds described herein include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; other cellulose polymers such as sodium carboxymethylcellulose; and other materials known to those of ordinary skill in the art. Other lamina forming materials that can be used for this purpose include, but are not limited to poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer.

While the compounds and formulations thereof and methods of use thereof are described herein with respect to use in humans, they are also suitable for animal, e.g., veterinary use. Certain illustrative non-human organisms include, but are not limited to non-human primates, canines, equines, felines, porcines, rodents, ungulates, lagomorphs, and the like.

The foregoing formulations and administration methods are intended to be illustrative and not limiting. It will be appreciated that, using the teaching provided herein, other suitable formulations and modes of administration can be readily devised.

Formulation into Food Products and/or Dietary Supplements.

When consumed, the compounds described herein are hydrolyzed to produce 1) ketone bodies or immediate precursors to ketone bodies, 2) lactate or immediate precursors to lactate, or 3) propinoyl 1-carninte which can provide a calorie source that can be classified as a food and can form part of a food product.

A food product is an edible material composed primarily of one or more of the macronutrients protein, carbohydrate and fat, which is used in the body of an organism (e.g., a mammal) to sustain growth, repair damage, aid vital processes or furnish energy. A food product may also contain one or more micronutrients such as vitamins or minerals, or additional dietary ingredients such as flavorants and colorants.

Examples of food products into which the compounds described herein or compositions/formulations thereof may be incorporated as an additive include, but are not limited to snack bars, meal replacement bars, cereals, confectionery and probiotic formulations including, but not limited to yoghurts.

Examples of beverages and drinks include, but are not limited to, soft beverages, energy drinks, dry drink mixes, nutritional beverages, meal or food replacement drinks, compositions for rehydration (for instance during or after exercise), and teas (e.g., herbal teas) for infusion or herbal blends for decoction in water.

In certain embodiments a composition for rehydration typically comprises water, a sugar (or non-sugar sweetener), carbohydrate and one or more of the compounds described herein. In certain embodiments the composition may also comprise suitable flavorings, colorants and preservatives, as will be appreciated by one of skill in the art. The carbohydrate sugar, when present, can provide an energy source, and suitable sugars are known, including glucose and trehalose. In certain embodiments a meal or food replacement drink may be of the type commonly advocated for use in weight loss regimens. Such drink formulations typically comprise appropriate quantities of one or more macronutrients, e.g., sources of protein, fat and/or carbohydrate, together with optional additional ingredients such as solubilizing agents, preservatives, sweetening agents, flavoring agents and colorants.

A nutraceutical is a food ingredient, food supplement or food product that is considered to provide a medical or health benefit, including the prevention and treatment of disease. In general, a nutraceutical is specifically adapted to confer a particular health benefit on the consumer. In various embodiments a nutraceutical typically comprises a micronutrient such as a vitamin, mineral, herb, and/or phytochemical at a higher level than would be found in a corresponding regular (natural) food product. That level is typically selected to optimize the intended health benefit of the nutraceutical when taken either as a single serving or as part of a diet regimen or course of nutritional therapy. In certain embodiments the level would be a level effective to reduce plasma levels of fatty acids.

A functional food is a food that is marketed as providing a health benefit beyond that of supplying pure nutrition to the consumer. A functional food typically incorporates an ingredient such as a micronutrient as mentioned above, that confers a specific medical or physiological benefit other than a nutritional effect. A functional food typically carries a health claim on the packaging.

In certain embodiments a nutraceutical or functional food product typically contains the compounds described herein in an amount effective to lower plasma levels of free fatty acids in a subject. In certain embodiments a nutraceutical or functional food product typically contains the compounds described herein in an amount effective to lower blood glucose. More typically the nutraceutical or functional food product contains the compounds in an amount effective to suppress appetite, and/or to induce weight loss in a subject.

A dietary supplement is a product that is intended to supplement the normal diet of a subject (e.g., a human subject) and which contains a dietary ingredient such as a vitamin, mineral, herb or other botanical product, or amino acid. A dietary supplement is typically presented in unit dosage format and is designed for consumption with, before or after food but not in place of food. A dietary supplement is thus often presented as a tablet or capsule, or as dried powder or granules for sprinkling over food or adding to water or a beverage.

In certain embodiments one or more of the compounds described herein (e.g., compounds according to any one of Formulas I to VI are provided in a formulation wherein the formulation further comprises a ketone salt and/or a ketone free acid. In certain embodiments the formulation comprises a ketone salt. In certain embodiments the formulation comprises a ketone salt of the same compound(s) described herein (e.g., Formulas I-VI). In certain embodiments the ketone salt is a ketone salt of bis hexanoyl (R)-1,3-butanediol. In certain embodiments the formulation comprises the compound(s) described herein and the ketone salt in a ratio ranging from about 0.5:1 wt/wt (compound:ketone salt) to about 3:1 (compound:ketone salt), or from about 1:1 wt/wt (compound:ketone salt) to about 2:1 wt/wt (compound:ketone salt). In certain embodiments the formulation comprises the compound(s) described herein and the ketone salt in a ratio ranging of about 2:1 wt/wt (compound:ketone salt).

In certain embodiments the formulation comprises a ketone free acid. In certain embodiments the formulation comprises a ketone free acid of bis hexanoyl (R)-1,3-butanediol. In certain embodiments the formulation comprises one or more compound(s) described herein and the ketone free acid in a ratio ranging from about 0.5:1 wt/wt (compound:ketone free acid) to about 3:1 (compound:ketone free acid), or from about 1:1 wt/wt (compound:ketone free acid) to about 2:1 wt/wt (compound:ketone free acid). In certain embodiments the formulation comprises one or more compound(s) described herein and the ketone free acid in a ratio of about 2:1 wt/wt (compound:ketone free acid).

In certain embodiments the formulation further comprises citric acid and/or malic acid. In certain embodiments the formulation further comprises a flavoring (e.g., a natural flavoring such as raspberry flavoring). In certain embodiments the formulation further comprises a sweetener. In certain embodiments the sweetener comprises monk fruit extract. In certain embodiments the formulation comprises a preservative (e.g., potassium sorbate, sodium benzoate, etc.).

### Kits.

In various embodiments, one or more compound(s) described herein and/or formulations thereof described herein thereof can be enclosed in multiple or single dose containers. The enclosed agent(s) can be provided in kits, for example, including component parts that can be assembled for use. For example, an active agent in lyophilized form and a suitable diluent may be provided as separated components for combination prior to use. A kit may include an active agent and a second therapeutic agent for co-administration. The active agent and second therapeutic agent may be provided as separate component parts. A kit may include a plurality of containers, each container holding one or more unit dose of the compounds. The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, medipads, creams, and the like for topical administration, e.g., as described herein.

In certain embodiments, a kit is provided wherein the kit comprises one or more compound(s) described herein and/or formulations/compositions thereof, or pharmaceutically acceptable salt or solvate of the enantiomer(s) preferably provided as a pharmaceutical composition and in a suitable container or containers and/or with suitable packaging; optionally one or more additional active agents, which if present are preferably provided as a pharmaceutical composition and in a suitable container or containers and/or with suitable packaging; and optionally instructions for use, for example written instructions on how to administer the compound or compositions.

As with any pharmaceutical product, the packaging material(s) and/or container(s) are designed to protect the stability of the product during storage and shipment. In addition, the kits can include instructions for use or other informational material that can advise the user such as, for example, a physician, technician, or patient, regarding how to properly administer the composition(s) as prophylactic, therapeutic, or ameliorative treatment of the disease of concern. In some embodiments, instructions can indicate or suggest a dosing regimen that includes, but is not limited to, actual doses and monitoring procedures.

In some embodiments, the instructions can include informational material indicating that the administering of the compositions can result in adverse reactions including but not limited to allergic reactions such as, for example, anaphylaxis. The informational material can indicate that allergic reactions may exhibit only as mild pruritic rashes or may be severe and include erythroderma, vasculitis, anaphylaxis, Steven-Johnson syndrome, and the like. In certain embodiments the informational material(s) may indicate that anaphylaxis can be fatal and may occur when any foreign protein is introduced into the body. In certain embodiments the informational material may indicate that these allergic reactions can manifest themselves as urticaria or a rash and develop into lethal systemic reactions and can occur soon after exposure such as, for example, within 10 minutes. The informational material can further indicate that an allergic reaction may cause a subject to experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, eosinophilia, or a combination thereof.

While the instructional materials typically comprise written or printed materials they are not limited to such. Any medium capable of storing such instructions and communicating them to an end user is contemplated herein. Such media include but are not limited to electronic storage media (e.g., magnetic discs, tapes, cartridges, chips), optical media (e.g., CD ROM), and the like. Such media may include addresses to internet sites that provide such instructional materials.

### EXAMPLES

In order that the disclosure described herein may be more fully understood, the following examples are set forth. It should be understood that these examples are for illustrative purposes only and are not to be construed as limiting this disclosure in any manner.

The compounds of the disclosure may be made according to standard chemical practices or as described herein. Throughout the following synthetic schemes and in the descriptions for preparing compounds of Formulae (I), and (II), the following abbreviations are used:

### Abbreviations

BnBr=Benzyl bromide
DCC=N,N′-dicyclohexylcarbodiimide
DCM=dichloromethane
DMAP=dimethylamino pyridine
DMF=dimethylformamide
LAH=lithium aluminum hydride
MeOH=methanol
Pd/C=Palladium on carbon
TEA=triethylamine
TfOH=triflic acid

### Example 1. Synthesis of Compounds

All the specific and generic compounds, and the intermediates disclosed in the following Schemes for making those compounds, are considered to be part of the disclosure disclosed herein.

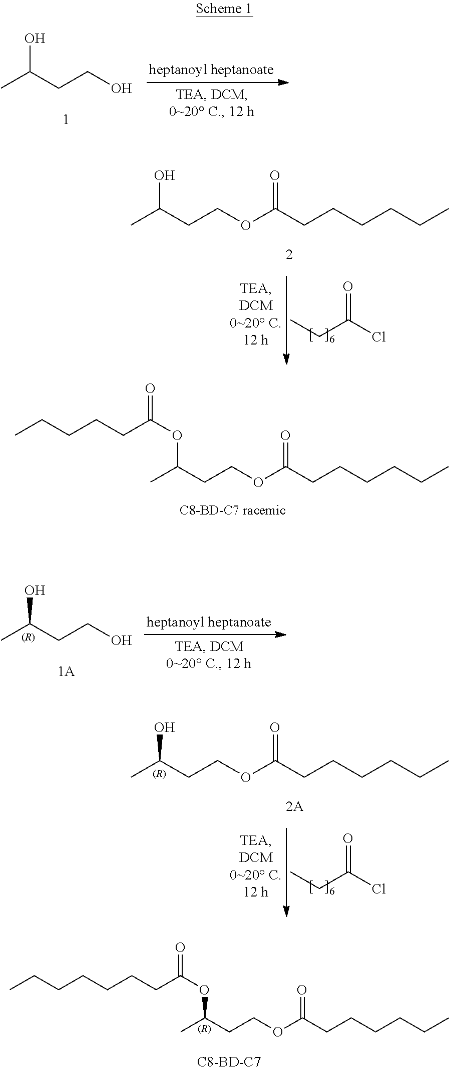

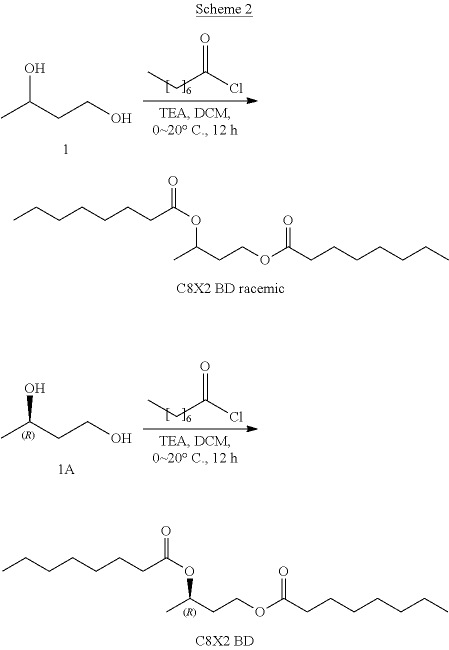

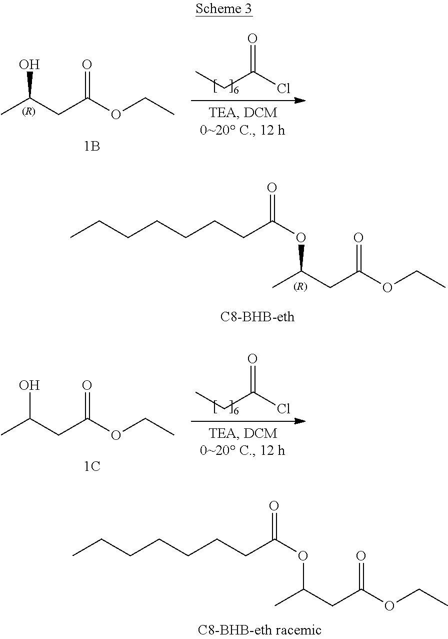

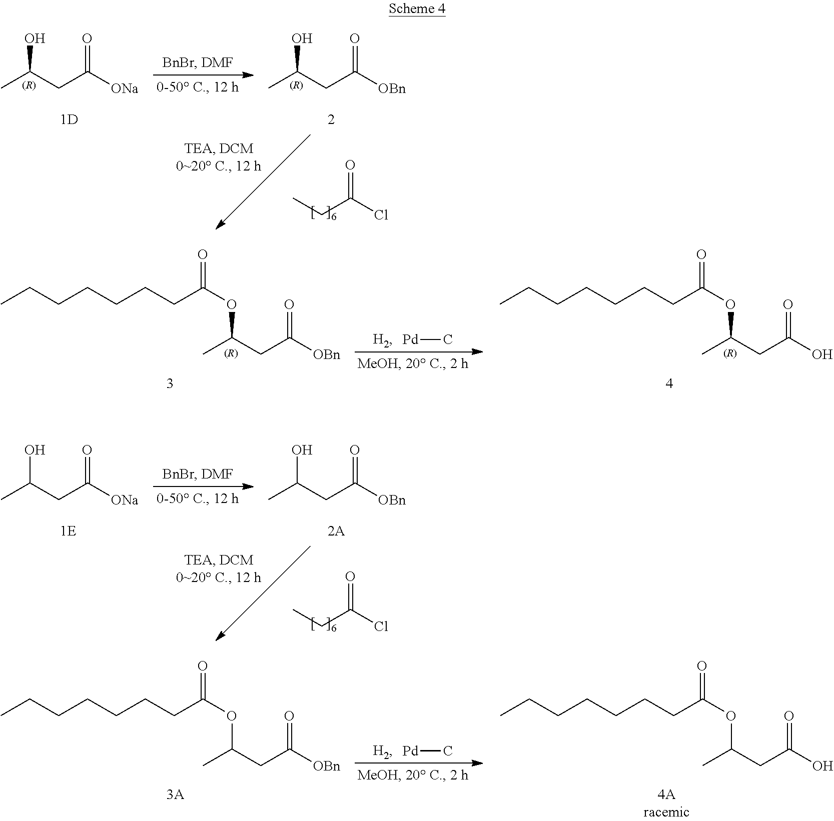

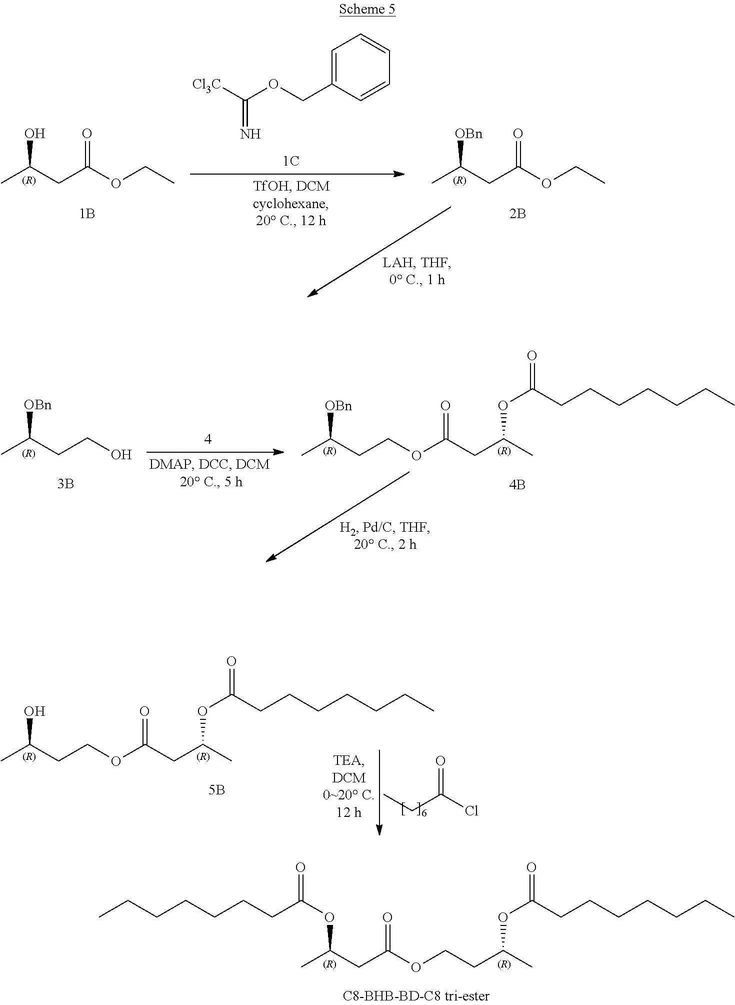

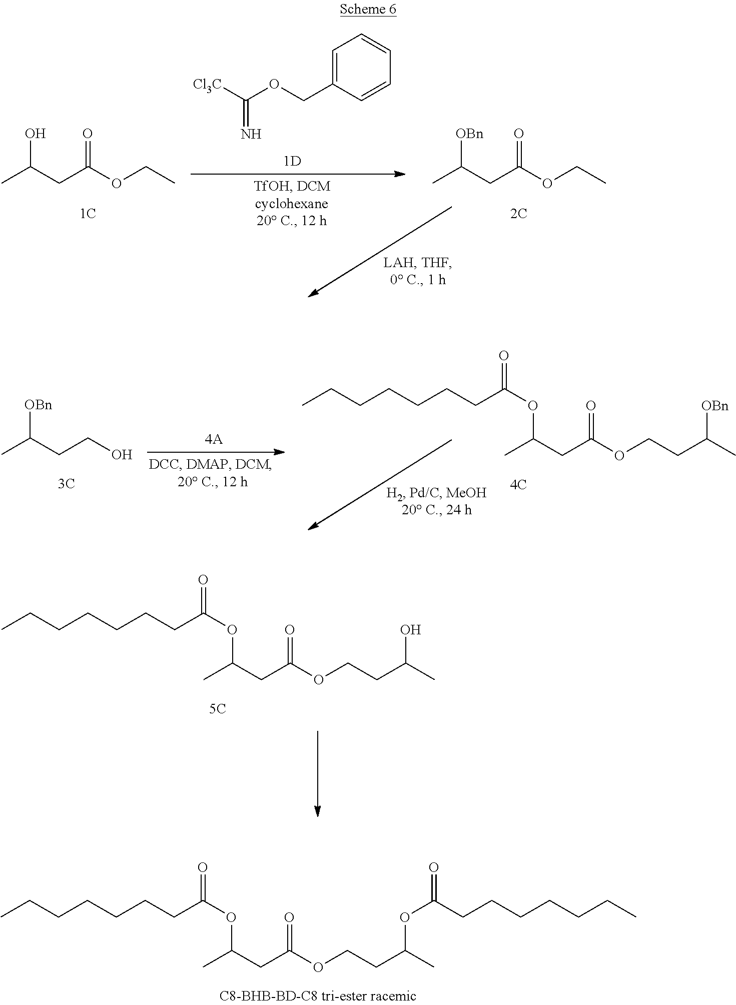

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound according to Formula I or Formula II: 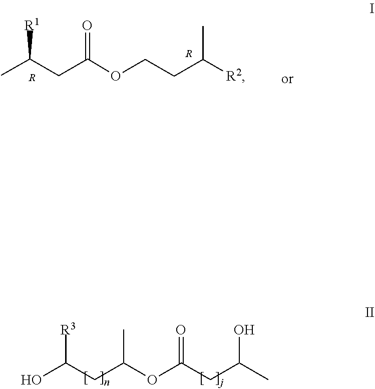
wherein:
R¹ is OH or C(4-14) saturated fatty acid;
R² is OH or C(4-14) saturated fatty acid;
at least one of R¹ or R² is said saturated fatty acid;
R³ is H, CH₃, or O;
n is 0 or 1;
j is 0 or 1; and
when R³ is O, a is a double bond.

2. The compound of claim 1, wherein said compound comprises a compound according to Formula I:

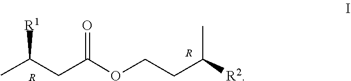

3. The compound according to any one of claims 1-2, wherein R¹ is OH.

4. The compound of claim 3, wherein R² is a C(4-10) fatty acid.

5. The compound of claim 4, wherein R² is a C(4-6) fatty acid.

6. The compound of claim 5, wherein said compound is a compound according to the formula:

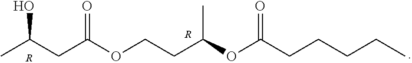

7. The compound according to any one of claims 1-2, wherein R² is OH.

8. The compound of claim 7, wherein R¹ is a C(4-10) fatty acid.

9. The compound of claim 8, wherein R¹ is a C(4-6) fatty acid.

10. The compound of claim 9, wherein said compound is a compound according to the formula:

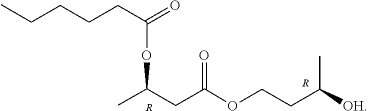

11. The compound according to any one of claims 1-2, wherein:
R¹ is a C(4-14) saturated fatty acid; and
R² is a C(4-14) saturated fatty acid.

12. The compound of claim 11, wherein said compound is a compound according to the formula:

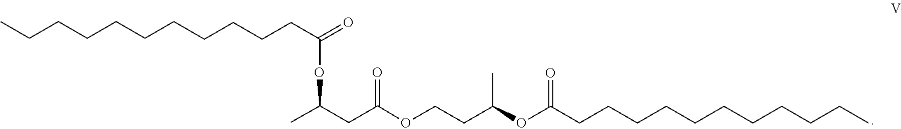 [SMILES: CCCCCCCCCCCCC(=O)O[C@H](C)CC(=O)OCC[C@@H](C)OC(=O)CCCCCCCCCCC]

13. The compound of claim 1, wherein said compound comprises a compound according to Formula II: 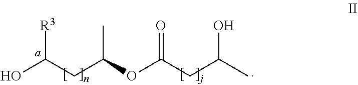
wherein at least one of n or j is 1.

14. The compound of claim 13, wherein n is 1, and j is 1, and said compound comprises a compound according to the formula:

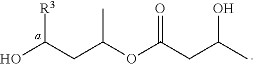

15. The compound of claim 13, wherein when n is 0, j is 1 and when j is 0, n is 1.

16. The compound of claim 15, wherein n is 0, and j is 1, and said compound comprises a compound according to the formula:

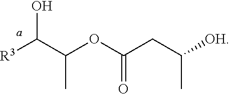

17. The compound of claim 16, wherein R³ is H or CH₃.

18. The compound of claim 16, wherein said compound comprises a compound according to the formula:

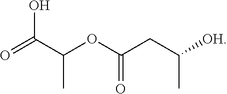

19. The compound of claim 15, wherein n is 1, and j is 0 and said compound comprise a compound according to the formula:

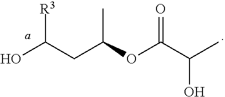

20. The compound of claim 19, wherein said compound comprises a compound according to the formula:

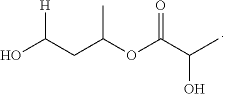

21. The compound of claim 19, wherein said compound comprises a compound according to the formula:

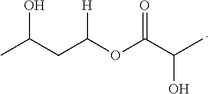

22. The compound of claim 19, wherein said compound comprises a compound according to the formula:

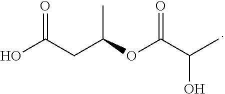

23. A compound according to Formula III, Formula IV, Formula V, or Formula VI: 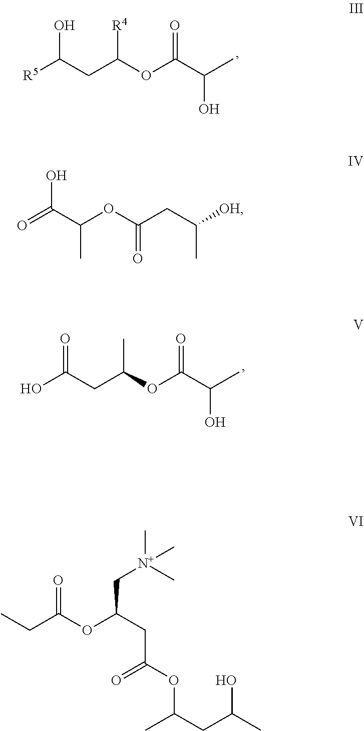
wherein:
R⁴ is H or CH₃;
R⁵ is H or CH₃; and
when R⁴ is H, R⁵ is CH₃ and when R⁴ is CH₃, R⁵ is H.

24. The compound of claim 23, wherein said compound comprises a compound according to the formula:

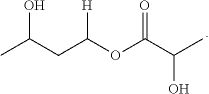

25. The compound of claim 23, wherein said compound comprises a compound according to the formula:

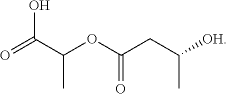

26. The compound of claim 23, wherein said compound comprises a compound according to the formula:

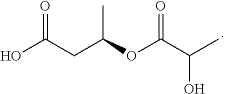

27. The compound of claim 23, wherein said compound comprises a compound according to the formula:

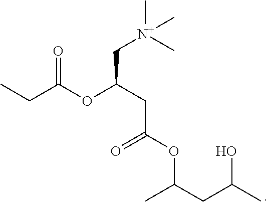 [SMILES: CCC(=O)O[C@H](CC(=O)OC(C)CC(C)O)C[N+](C)(C)C]

28. A pharmaceutical formulation comprising a compound according to any one of claims 1-27, and a pharmaceutically acceptable carrier.

29. The formulation of claim 28, wherein said formulation is for administration via a modality selected from the group consisting of intraperitoneal administration, topical administration, oral administration, inhalation administration, transdermal administration, subdermal depot administration, and rectal administration.

30. An ingestible composition that comprises a compound according to any one of claims 1-27, and a dietetically or pharmaceutically acceptable carrier.

31. The composition of claim 30, wherein said composition comprises a dietetically acceptable carrier.

32. The composition of claim 31, wherein said composition comprises a food product, a beverage, a drink, a food supplement, a dietary supplement, a functional food, or a nutraceutical.

33. A food supplement comprising a compound according to any one of claims 1-27.

34. A composition comprising:
a compound according to any one of claims 1-27; and
one or more components of a ketogenic diet.

35. A method of preventing the onset, slowing the progression of, and/or treating heart failure, said method comprising:
administering to a subject in need thereof an effective amount of a compound according to any one of claims 1-27, and/or pharmaceutical formulation according to of claim 28.

36. A method of treating dementia or other neurocognitive disorder, said method comprising:
administering to a subject in need thereof an effective amount of a compound according to any one of claims 1-27, and/or pharmaceutical formulation according to claim 28.

37. A method of preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease, said method comprising:
administering to a subject in need thereof a compound according to any one of claims 1-27, and/or pharmaceutical formulation according to claim 28 in an amount sufficient to prevent or delay the onset of a pre-Alzheimer's cognitive dysfunction, and/or to ameliorate one or more symptoms of a pre-Alzheimer's cognitive dysfunction, and/or to prevent or delay the progression of a pre-Alzheimer's cognitive dysfunction to Alzheimer's disease.

38. The method of claim 37, wherein said method is a method of preventing or delaying the transition from a cognitively asymptomatic pre-Alzheimer's condition to a pre-Alzheimer's cognitive dysfunction.

39. A method of reducing epileptiform activity in the brain of a subject, said method comprising administering to a subject an effective amount of a compound according to any one of claims 1-27, and/or pharmaceutical formulation according to claim 28.

40. A method for treating, in a subject, one or more of epilepsy, Parkinson's disease, heart failure, traumatic brain injury, stroke, hemorrhagic shock, acute lung injury after fluid resuscitation, acute kidney injury, myocardial infarction, myocardial ischemia, diabetes, glioblastoma multiforme, diabetic neuropathy, prostate cancer, amyotrophic lateral sclerosis, Huntington's disease, cutaneous T cell lymphoma, multiple myeloma, peripheral T cell lymphoma, HIV, Niemann-Pick Type C disease, age-related macular degeneration, gout, atherosclerosis, rheumatoid arthritis and multiple sclerosis comprising:
administering to a subject in need thereof an effective amount of a compound according to any one of claims 1-27, and/or pharmaceutical formulation according to claim 28.

41. A method of treating a condition which is caused by, exacerbated by or associated with elevated plasma levels of free fatty acids in a human or animal subject, which method comprises administering to a subject in need thereof an effective amount of a compound according to any one of claims 1-27, and/or pharmaceutical formulation according to claim 28.

42. A method of treating a condition wherein weight loss or weight gain is implicated, which method comprises administering to a subject in need thereof an effective amount of a compound according to any one of claims 1-27, and/or pharmaceutical formulation according to claim 28.

43. A method of suppressing appetite, treating obesity, promoting weight loss, maintaining a healthy weight or decreasing the ratio of fat to lean muscle, said method comprising administering to a subject in need thereof an effective amount of a compound according to any one of claims 1-27, and/or pharmaceutical formulation according to claim 28.

44. A method of preventing or treating a condition selected from cognitive dysfunction, a neurodegenerative disease or disorder, muscle impairment, fatigue and muscle fatigue, said method comprising administering to a subject in need thereof an effective amount of a compound according to any one of claims 1-27, and/or pharmaceutical formulation according to claim 28.

45. A method of treating a subject suffering from a condition selected from diabetes, hyperthyroidism, metabolic syndrome X, or for treating a geriatric patient, said method comprising administering to a subject in need thereof an effective amount of a compound according to any one of claims 1-27, and/or pharmaceutical formulation according to claim 28.

46. A method of treating, preventing, or reducing the effects of, neurodegeneration, free radical toxicity, hypoxic conditions or hyperglycemia, said method comprising administering to a subject in need thereof an effective amount of a compound according to any one of claims 1-22, and/or pharmaceutical formulation according to claim 28.

47. A method of treating, preventing, or reducing the effects of, neurodegeneration, said method comprising administering to a subject in need thereof an effective amount of a compound according to any one of claims 1-27, and/or pharmaceutical formulation according to claim 28.

48. A method of preventing or treating a neurodegenerative disease or disorder selected from Alzheimer's disease, Parkinson's disease, amyotrophic lateral sclerosis, epilepsy, astrocytoma, glioblastoma and Huntington's chorea, said method comprising administering to a subject in need thereof an effective amount of a compound according to any one of claims 1-27, and/or pharmaceutical formulation according to claim 28.

49. A method of promoting alertness or improving cognitive function in a subject, said method comprising administering to a subject in need thereof an effective amount of a compound according to any one of claims 1-22, and/or pharmaceutical formulation according to claim 28.
